(19) United States
(12) Patent Application Publication
(10) Pub. No.: US 2004/0229360 A1
(43) Pub. Date: Nov. 18, 2004

(54) Cardiac-preferred genetic alteration of transgenic rabbits

(76) Inventor: Jeffrey Robbins, Cincinnati, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 10/798,037

(22) Filed: Mar. 11, 2004

Related U.S. Application Data
(60) Provisional application No. 60/454,947, filed on Mar. 13, 2003.

(51) Int. Cl. C12N 5/06; C12N 15/85

(52) U.S. Cl. 435/455; 435/325

(57) ABSTRACT

The methods and compositions of the present invention find use in altering cardiac-preferred expression in transgenic animals. The compositions of the invention include animal cells, transgenic animals, and transgenic rabbits. The transgenic animals of the invention exhibit altered cardiac preferred expression of a heterologous nucleotide sequence. The methods allow generation of transgenic animals with altered cardiac preferred expression of the heterologous nucleotide sequence. In particular, the invention provides a method for altering the susceptibility of a transgenic animal to cardiopathy. A transgenic animal of the invention finds use in identifying anti-cardiopathic compounds.

### CROSS-REFERENCE TO RELATED APPLICATIONS

This application claims priority to and benefit of U.S. Provisional Application No. 60/454,947, filed on Mar. 13, 2003, which is herein incorporated by reference in its entirety.

### GOVERNMENT GRANT INFORMATION

This invention was made with Government support under NIH Grant Nos. 1ROHL56370 and PO1 HL53218-01. The United States Government has certain rights in this invention.

### FIELD OF THE INVENTION

The present invention relates to the field of regulation of tissue-preferred gene expression.

### BACKGROUND OF THE INVENTION

A variety of human diseases and conditions which are manifested by cardiac abnormalities or cardiac dysfunction can lead to heart failure. Heart failure is a physiological condition in which the heart fails to pump enough blood to meet the circulatory requirements of the body. The study of such diseases and conditions in genetically diverse humans is difficult and unpredictable. Therefore, there is a need for a model system which facilitates the study of the mechanisms and causes of cardiac diseases and conditions as well as the identification of potential therapeutic targets.

The development of transgenic animal technology has provided significant advances for obtaining more complete information about complex systems in vivo. By manipulating the expression of a gene or genes in vivo, it is possible to gain insight into the roles of such genes in a particular system or to study aspects of the system in a genetically controlled environment.

While successful transgene experiments have been performed in a number of large and small animal species, the mouse has been the animal of choice for cardiovascular studies. See, for example, U.S. Pat. No. 6,353,151, herein incorporated by reference. Cardiac preferred transgenesis has been used to establish structure-function relationships between the presence or absence of a particular protein (or its mutated form) and normal or abnormal function at the molecular, cellular, and physiological levels. However, there are significant differences between mouse models and human disease presentation, particularly with regard to familial hypertrophic cardiomyopathies (FHC). Mouse models of FHC exhibit depressed contractility, absence of cardiac sudden death, a relatively small increase in ventricular mass, and a gender bias not found in human presentations of FHC. Mice hearts beat ten times faster than human hearts. The models diverge at the molecular level also; the α-myosin heavy chain isoform predominates in the adult mouse ventricle while the β-myosin heavy chain isoform predominates in adult human ventricles.

Thus, development of a transgenic model system is desirable for use in studying heart disease and conditions. It is of importance to develop a transgenic, cardiac-preferred expression model in system closely related to the human heart at the molecular, biochemical, and physiological levels. It is of particular importance to develop a model transgenic system for use in studying familial hypertrophic cardiomyopathies.

### SUMMARY OF THE INVENTION

Compositions and methods for cardiac-preferred expression of heterologous nucleotide sequences are provided. Compositions of the invention include animals, particularly transgenic rabbits, and animal cells. Animals, transgenic rabbits, and animal cells of the invention comprise an expression cassette comprising a promoter capable of initiating tissue-preferred transcription, particularly cardiac-preferred transcription, operably linked to a heterologous nucleotide sequence. In an embodiment the promoter is capable of initiating ventricle-preferred transcription. In an embodiment the promoter is capable of initiating atria-preferred transcription. In animals and transgenic rabbits of the invention, expression of the heterologous nucleotide sequence is altered, particularly in cardiac tissue. In an embodiment, expression of the heterologous nucleotide sequence is ventricle-preferred. In an embodiment, expression of the heterologous nucleotide sequence is atria-preferred. In an embodiment, the transgenic rabbit's genome comprises a promoter capable of initiating ventricle-preferred transcription operably linked to a heterologous nucleotide sequence encoding the rabbit α-myosin heavy chain polypeptide. Transgenic rabbits comprising a heterologous nucleotide sequence encoding the rabbit α-myosin heavy chain polypeptide operably linked to a promoter capable of initiating ventricle-preferred transcription exhibit altered myosin-isoform expression.

Methods for modulating expression of heterologous nucleotide sequences in animals are provided. The animal's susceptibility to various cardiopathies, including but not limited to, cardiomyopathies, may be altered by the methods of the invention. Cardiomyopathies include, but are not limited to, familial hypertrophic cardiomyopathy, dilated cardiomyopathies, peripartum cardiomyopathy, and restrictive cardiomyopathies. In an embodiment, the animal exhibits increased susceptibility to cardiopathy. In one embodiment, the animal exhibits decreased susceptibility to cardiopathy. Expression cassettes comprising a promoter with a nucleotide sequence capable of initiating tissue-preferred, particularly cardiac-tissue preferred, transcription in the animal are developed. The promoter is operably linked to a heterologous nucleotide sequence of interest. An expression cassette comprising a promoter capable of initiating cardiac-preferred expression operably linked to a heterologous nucleotide sequence of interest is used to generate a transgenic animal. The genome of the animal incorporates at least one expression cassette comprising the promoter and the heterologous nucleotide sequence. The heterologous nucleotide sequence is preferentially expressed in a cardiac tissue. Expression of the heterologous nucleotide sequence can be assessed by any method known to one skilled in the art. In an embodiment the heterologous nucleotide sequence encodes a myocardial component such as, but not limited to, α-myosin heavy chain, β-myosin heavy chain, essential myosin light chain-1, actin, catecholamine receptors, and glycogen synthase 3-β.

Methods for identifying anti-cardiopathic compounds are provided. In an embodiment at least two transgenic rabbits whose genomes comprise at least one stably incorporated expression cassette comprising a promoter capable of initiating cardiac-preferred transcription operably linked to a heterologous nucleotide sequence are provided. A compound of interest is administered to the first rabbit. The first and second rabbits are incubated for a period of time. A cardiopathic phenotype is monitored in both rabbits. Cardiopathic phenotypes include, but are not limited to, mortality, cardiac myocyte disarray, interstitial fibrosis, systolic dysfunction, diastolic dysfunction, left ventricular hypertrophy, cardiac mass abnormalities, right ventricular outflow tract obstruction, morphological changes, cellular degeneration, and hyper-contractility.

In an embodiment, the invention provides a transgenic rabbit comprising at least one expression cassette comprising a promoter operably linked to a heterologous nucleotide sequence, wherein the transgenic nucleotide sequence is set forth in SEQ ID NO:5. In an aspect of the invention, the transgenic rabbit exhibits altered myosin isoform expression in ventricle tissue.

The invention further provides kits comprising a transgenic animal, particularly a transgenic rabbit comprising an expression cassette comprising a promoter having a nucleotide sequence set forth in SEQ ID NO:1 or SEQ ID NO:2 or a fragment or variant thereof and a heterologous nucleotide sequence operably linked to the promoter.

In an embodiment, the invention provides a kit for performing a method of altering expression of a heterologous nucleotide sequence in a rabbit comprising a transgenic rabbit of the invention.

In an embodiment, the invention provides a kit for performing a method of altering expression of a heterologous nucleotide sequence in an animal comprising an expression cassette comprising a promoter, wherein said promoter comprises a nucleotide sequence set forth in SEQ ID NO:1 or SEQ ID NO:2, or a fragment or variant thereof.

In another embodiment, the invention provides a kit for altering an animal's susceptibility to cardiopathy. Such a kit comprises a transgenic animal of the invention, particularly a transgenic rabbit of the invention. In an aspect of the invention, the kit further comprises a non-transgenic animal, particularly a non-transgenic rabbit.

In an embodiment, the invention provides a kit for identifying anti-cardiopathic compounds. Such a kit comprises a first and second transgenic rabbit of the invention.

### BRIEF DESCRIPTION OF THE DRAWINGS

FIG. 1 depicts cardiac preferred expression of the CAT reporter gene operably linked to the β-myosin heavy chain promoter (SEQ ID NO:2). Tissues were obtained from multiple animals in the F2 generations of four stable transgenic lines. Two, fourteen, twenty-four, and thirty-nine copies of the transgene are present in the genomes of transgenic lines 415 (white bars), 428L (right slash bars), 428M (left slash bars), and 428H (hatched bars), respectively. Panel A presents the results of CAT ELISAs performed on tissues obtained from the apex, left ventricle (LV free), septum, right ventricle (RV free), left atria, and right atria. Panel B presents the results of CAT ELISAs performed on diaphragm (Dia) and soleus (Sol) tissues. The diaphragm contains mixed fiber types while the soleus is a slow skeletal muscle. Panel C presents the results of CAT ELISAs performed on bicep muscle (Bic), tibialis muscle (Tibi), masseter muscle (Mass), tongue (Tong), stomach (Stom), small intestine (S. Inte), lung (Lun), liver (Liv), and spleen (Sple). CAT activity is presented in pg/μg protein.

FIG. 2, Panel A presents the results of RNA analysis of cardiac tissue from transgenic (TG) and nontransgenic (NTG) rabbits. Right atria (RA), left atria (LA), right ventricles (RV), and left ventricles (LV) were isolated from a control animal and a transgenic rabbit carrying the β-myosin heavy chain promoter (SEQ ID NO:2) operably linked to the α-myosin heavy chain coding sequence (SEQ ID NO:3). RNA was isolated from the tissues and dotted on to a membrane as described elsewhere herein. The dot blots were probed with radiolabeled oligonucleotides specific to either the α-myosin heavy chain gene (α) or the β-myosin heavy chain gene (β) as described elsewhere herein.

FIG. 2, Panel B presents the results of protein analysis of cardiac tissue from a control animal and a transgenic rabbit carrying the β-myosin heavy chain promoter (SEQ ID NO:2) operably linked to the α-myosin heavy chain coding sequence (SEQ ID NO:3). Lanes 1 and 2 contain samples from the right atria (RA) and left ventricle (LV) of the control rabbit. Lanes 3, 4, 5, and 6 contain total protein samples from the right atria (RA), left atria (LA), right ventricle (RV), and left ventricle (LV) of the transgenic rabbit. The proteins were resolved by electrophoreses on polyacrylamide gels and transferred to nylon membranes. The membranes were probed with α-myosin heavy chain specific antibodies to detect the α-myosin heavy chain polypeptide.

FIG. 3 presents the results of actin-activated ATPase experiments performed on cardiomyocytes obtained from transgenic ventricles, non-transgenic ventricles, and rabbit atria. The actin concentration is indicated in μmol on the x-axis. Inorganic phosphate (Pi) is indicated in nmol Pi produced per minute per mg of total protein on the y-axis. Data obtained from non-transgenic ventricles (primarily P-MHC) are indicated with solid diamonds. Data obtained from transgenic ventricles (α-MHC/β-MHC) are indicated with open squares. Data obtained from atria (α-MHC) are indicated with solid triangles.

FIG. 4 depicts an implanted device used to alter cardiac pace and data obtained from animals implanted with such a device. An implanted pacemaker device is shown in Panel A. Echocardiograms obtained from transgenic and non-transgenic rabbits are shown in Panel B and Panel C, respectively. Experimental details are described elsewhere herein.

FIG. 5 presents the shortening fractions of transgenic (white squares) and non-transgenic (solid diamonds) rabbits determined prior to implantation of a pacemaker (Pre-op) and prior to initiating the indicated paces. Shortening fractions were determined before pacing the animals to 300 beats per minute (Pre-300), 340 beats per minute (Pre-340), 380 beats per minute (Pre-380), and ten days after pacing the animals to 380 beats per minute (Final). Experimental details are described elsewhere herein.

FIG. 6 presents the heart rate corrected velocity of circumferential shortening (V_{CFC}) and left ventricular end-systolic meridional wall stress (WS, measured in g/cm²) of transgenic (hatched circles) and nontransgenic (left slash circles) rabbits after undergoing the pacing protocol described above. The large circles indicate the mean values for each group. The data are superimposed on numbers derived from healthy human subjects (empty circles); see Kimball et al. (1991) Am. J. Cardiol. 68:1383-1387.

### DETAILED DESCRIPTION OF THE INVENTION

The present invention provides for cardiac-preferred expression of transgenes in animals. Compositions of the invention include transgenic rabbits comprising a promoter of the invention operably linked to a nucleotide sequence of interest. The invention provides methods for altering expression of a nucleotide sequence of interest in an animal. Expression of the nucleotide sequences of interest may alter a transgenic animal's susceptibilities to cardiopathies. Further, the invention provides methods for identifying anti-cardiopathic compounds.

The invention relates to compositions and methods drawn to the rabbit α-myosin heavy chain promoter (SEQ ID NO: 1), β-myosin heavy chain promoter (SEQ ID NO:2), the α-myosin heavy chain (SEQ ID NO:3), and methods of their use. An animal cell or animal of the invention is stably transformed with an expression cassette comprising the cardiac-preferred promoters set forth in SEQ ID NO: 1 or 2 operably linked to a heterologous nucleotide sequence. The promoter sequences are useful for expressing operably linked sequences in a tissue-preferred, preferably cardiac-tissue preferred expression pattern. In an embodiment the rabbit α-myosin heavy chain promoter (SEQ ID NO: 1) is an atria-preferred promoter. In an embodiment, the rabbit β-myosin heavy chain promoter (SEQ ID NO:2) is a ventricle-preferred promoter.

Transgenic embryos of the transgenic rabbits of the invention were deposited with the Patent Depository of the American Type Culture Collection (ATCC), Manassas, Va. on ______, ______ and assigned Patent Deposit No. ______. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

By “stably transformed” is intended that the nuclear genome of the animal cell or the nuclear genome of at least one cell of the animal has incorporated at least one copy of the transgene. A transgenic animal of the invention comprises at least one stably transformed cell comprising the nucleotide sequence of interest. In an embodiment, the genome of a germ-line cell of a transgenic animal comprises the nucleotide sequence of interest. The invention encompasses isolated or substantially purified nucleic acid or protein compositions. An “isolated” or substantially “purified” nucleic acid molecule, or biologically active portion thereof, is substantially free of other cellular material, or culture medium when produced by recombinant techniques or substantially free of chemical precursors or other chemicals when chemically synthesized. Preferably, an “isolated” nucleic acid molecule is free of sequences (preferably polypeptide encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

The promoters for the α-myosin heavy chain and β-myosin heavy chain genes may generally be isolated from the 5′ untranslated region flanking their respective transcription initiation sites. Methods for isolation of promoter regions are well known in the art. By “isolated” is intended that the promoter sequences have been determined and can be extracted by molecular techniques or synthesized by chemical means. In either instance, the promoter is removed from at least one of its flanking sequences in its native state.

Fragments and variants of the disclosed nucleotide sequences are also encompassed by the present invention. By “fragment” is intended a portion of the nucleotide sequence. Fragments of a nucleotide sequence may retain biological activity and drive expression, particularly tissue-preferred expression, more particularly cardiac-preferred expression, yet more particularly ventricle-preferred or atria-preferred expression. Alternatively, fragments of a nucleotide sequence that are useful as hybridization probes generally do not retain biological activity. Thus, fragments of a nucleotide sequence may range from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, up to about 5190 nucleotides for SEQ ID NO: 1; from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, 5200, 5250, 5300, 5350, 5400, 5500, 5600, 5700, 5800, 5900, 6000, 6100, 6200, 6300, 6400, 6500, 6600, 6700, 6800, 6900, up to about 6921 nucleotides for SEQ ID NO:2; from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, and up to about 5886 nucleotides for SEQ ID NO:3; from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 960, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, 5200, 5250, 5300, 5350, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000, 12100, 12200, 12300, 12400, 12500, 12600, 12700, 12800, and up to about 12,801 nucleotides for SEQ ID NO:5.

Thus a fragment of a nucleotide sequence for cardiac-preferred promoters may encode a biologically active portion of a cardiac tissue-preferred promoter, or it may be a fragment that can be used as a hybridization probe or PCR primer using methods disclosed below. A biologically active portion of a cardiac tissue preferred promoter can be prepared by isolating a portion of the promoter nucleotide sequence disclosed herein, and assessing the activity of the portion of the promoter. Nucleic acid molecules that are fragments of a cardiac-preferred promoter comprise 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, up to about 5190 nucleotides for SEQ ID NO:1; from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, 5200, 5250, 5300, 5350, 5400, 5500, 5600, 5700, 5800, 5900, 6000, 6100, 6200, 6300, 6400, 6500, 6600, 6700, 6800, 6900, up to about 6921 nucleotides for SEQ ID NO:2; from at least about 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 4500, 4550, 4600, 4650, 4700, 4750, 4800, 4850, 4900, 4950, 5000, 5050, 5100, 5150, 5200, 5250, 5300, 5350, 5400, 5500, 5600, 5700, 5800, 5900, 6000, 6100, 6200, 6300, 6400, 6500, 6600, 6700, 6800, 6900, or up to 6921 for SEQ ID NO:5.

By “variants” is intended substantially similar sequences. For nucleotide sequences, naturally occurring variants can be identified with the use of well-known molecular biology techniques, as, for example, with polymerase chain reactions (PCR) and hybridization techniques as outlined below. Variant nucleotide sequences also include synthetically derived nucleotide sequences, such as those generated for example, by using site-directed mutagenesis. Generally, variants of a particular nucleotide sequence of the invention will have at least 85%, generally at least 90%, 91%, 92%, 93%, 94%, preferably about 95%, 96%, 97%, and more preferably 98%, 99%, or more sequence identity to that particular nucleotide sequence as determined by sequence alignment programs described elsewhere herein using default parameters. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Co., New York) and the references cited therein.

Variant nucleotide sequences also encompass sequences derived from a mutagenic and recombinogenic procedure such as DNA shuffling. With such a procedure, one or more different promoter sequences including the promoter sequences disclosed herein, can be manipulated to create a new promoter sequence possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci.91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. 94:4504-4509; Crameri et al. (1998) Nature 391:288-291; Miyazaki (2002) Nucleic Acids Research 30:E139-9; Song et al. (2002) Appl. Environ. Microbiol. 68:6146-51; Hayes et al. (2002) Proc. Natl Acad Sci. 99:15926-31; Coco et al. (2001) Nature Biotechnol. 19:354-9; Kikuchi et al. (2000) Gene 243:133-7; and U.S. Pat. Nos. 5,606,793 and 5,837,458.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein “comparison window” makes reference to a contiguous and specified segment of a polynucleotide sequence, wherein the polynucleotide sequence in the comparison window may comprise additions or deletions (i.e. gaps) compared to the reference sequence (which does not comprise additions or deletions) for optimal alignment of the two sequences. Generally the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. For purposes of the present invention, comparison of nucleotide or protein sequences for determination of percent sequence identity to the sequences disclosed herein is preferably made using the GCG program GAP (Version 10.00 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Sequence comparison programs include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM 120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of I and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The nucleotide sequences disclosed herein can be used to isolate corresponding sequences from other organisms, particularly other mammals. In this manner, methods such as PCR, hybridization, and the like can be used to identify such sequences based on their sequence homology to the sequences set forth herein. Sequences isolated based on their sequence identity to the entire sequences set forth herein or to fragments thereof are encompassed by the present invention. Comparable promoter regions from other organisms, including other mammals, may be obtained by utilization of the coding or promoter sequences set forth herein.

In a PCR approach, oligonucleotide primers can be designed for use in PCR reactions to amplify corresponding DNA sequences from cDNA or genomic DNA extracted from any animal of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the promoter sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire promoter sequence disclosed herein, or one or more portions thereof, may be used as a probe capable of specifically hybridizing to corresponding promoter sequences. To achieve specific hybridization under a variety of conditions, such probes include sequences that are unique among cardiac-preferred promoter sequences and are preferably at least about 10 nucleotides in length, and most preferably at least about 20 nucleotides in length. Such probes may be used to amplify corresponding promoter sequences from a chosen animal by PCR. This technique may be used to isolate additional promoter sequences from a desired animal or as a diagnostic assay to determine the presence of the promoter sequences in an animal or animal cell.

Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with approximately 90% identity are sought, the Tₘ can be decreased 110° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). Thus, isolated sequences that have promoter activity and which hybridize under stringent conditions to the cardiac-preferred promoter sequences disclosed herein, or to fragments thereof, are encompassed by the present invention. Such sequences will be at least 85%, 90%, 95% to 98% homologous or more with the disclosed sequences. That is, the sequence identity of sequences may range, sharing at least 85%, 90%, 91%, 92%, 93%, 94%, 95%, 96%, 97%, 98%, 99% or more sequence identity.

Cardiac preferred promoters disclosed in the invention may be isolated from any animal, including but not limited to, rabbit, mouse, monkey, chimpanzee, dog, pig, goat, sheep, cat, and cow. It is recognized that any gene of interest can be operably linked to a promoter of the invention and expressed in cardiac tissue. By “cardiac tissue” is intended any tissue obtained from the heart, including but not limited to, tissues developmentally related to the heart. By “ventricle tissue” is intended any tissue obtained from any portion of either ventricle of the heart. By “atria tissue” is intended any tissue obtained from any portion of either atria of the heart.

General categories of genes of interest for the purposes of the present invention include for example, those genes involved in information, such as Zinc fingers, those involved in communication, such as kinases, and those involved in housekeeping, such as heat shock proteins. For example, genes of interest include but are not limited to, potassium channel genes, nitric oxide synthases, glycoprotein receptors, class 1 HLA, class 2 HLA, cathepsin B, cysteine aminopeptidases, acid gelatinases, trypsin-like endopeptidases, chymotrypsin-like endopeptidases, neutral gelatinases, angiotensin type-II receptors, myocardial sarcoplasmic reticulum Ca²⁺-ATPase, troponin T, troponin I, α-tropomyosin, TGF-β1, IGF-I, IGF-II, PDGF-B, prorenin, rennin, myosin binding protein C, ion channel genes, retinoic acid receptors, α-myosin heavy chains, β-myosin heavy chains, essential myosin light chains, actins, and sarcomere components.

It is recognized that the genes of interest vary for an atria-preferred promoter, such as the promoter set forth in SEQ ID NO:1, versus a ventricle-preferred promoter, such as the promoter set forth in SEQ ID NO:2.

The heterologous nucleotide sequence expressed by the promoters of the invention may be used for varying the phenotype of the heart. Various phenotypes of interest in cardiac tissue include, but are not limited to, hypertrophy; morphology, such as interventricular septal thickness; left ventricular-end systolic or end-diastolic dimensions; papillary muscle dimension; left-ventricular outflow tract obstruction; sarcomere structure, particularly alterations resulting in familial hypertrophic cardiomyopathy; alteration of myosin isoform expression, particularly resulting in altered susceptibility to cardiopathies; myofibril function; cardiopathic susceptibility; responsiveness to anti-cardiopathic compounds; receptor expression; heart rate; ventricular systolic pressure, ventricular diastolic pressure; aortic systolic pressure; aortic diastolic pressure; contractility; interstitial fibrosis; cardiomyocyte disarray; Ca²⁺ sensitivity; catecholine sensitivity; α-adrenergic sensitivity; beta-adrenergic sensitivity; angiotensin-converting enzyme inhibitor sensitivity; amiodarone sensitivity; lidocaine sensitivity; glycoprotein receptor antagonist sensitivity; anabolic steroid sensitivity; and the like.

These results can be achieved by providing expression of heterologous products or increased expression of endogenous products in cardiac tissue. Alternatively, the results can be achieved by providing for a reduction of expression of one or more endogenous products, particularly enzymes and cofactors in the cardiac tissue. These changes result in a change in phenotype of the transgenic animal. For example, the promoter sequences of the invention can be used to preferentially express the α-myosin heavy chain isoform in the ventricles and alter the myosin isoform expression pattern. Alternatively, the promoter sequences of the invention can be used to produce antisense mRNA complementary to the coding sequence of a cardiac protein, inhibit production of the protein, and alter expression of the heterologous nucleotide sequence. Alternatively, the promoter sequences of the invention can be used to produce small interfering RNAs.

Products of the heterologous nucleotide sequence include structural proteins, enzymes, cofactors, hormones, signaling proteins, and the like.

As noted, the heterologous nucleotide sequence operably linked to one of the promoters disclosed herein may be an antisense sequence for a targeted gene. Thus, with these promoters, antisense constructions complementary to at least a portion of the messenger RNA (mRNA) for a targeted sequence sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used. Thus, the promoter sequences disclosed herein may be operably linked to antisense DNA sequences to reduce or inhibit expression of a native protein in cardiac tissue.

By “promoter” or “transcriptional initiation region” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. It is recognized that having identified the nucleotide sequences for the promoter regions disclosed herein, it is within the state of the art to isolate and identify further regulatory elements in the 5′ untranslated region, typically downstream from the particular promoter regions identified herein. Thus, the promoter regions disclosed herein are generally further defined by comprising upstream regulatory elements such as those responsible for tissue and temporal expression of the coding sequence, enhancers and the like. Such elements are typically linked via a 5′ untranslated region, which may further modulate gene expression, to a coding region of interest. In the same manner, the promoter elements which enable expression in the desired tissue such as cardiac-tissue can be identified, isolated, and used with other core promoters to confirm cardiac-preferred expression. For genes in which the 5′ untranslated region does not affect cell specificity, alternative sources of 5′ untranslated leaders may be used in conjunction with these promoter elements.

The regulatory sequences of the present invention, when operably linked to a heterologous nucleotide sequence of interest and inserted into an expression vector, enable cardiac-preferred expression of the heterologous nucleotide sequence in the cardiac tissue of an animal stably transformed with this expression vector. By “cardiac-preferred” is intended that expression of the heterologous sequence is most abundant in cardiac tissue, while some expression may occur in other tissue types, particularly in tissues developmentally related to cardiac tissue. Cardiac-preferred expression of a heterologous nucleotide sequence of interest occurs at levels at least 1%, 5%, 10%, 20%, 30%, 40%, 50%, 60%, 70%, 80%, 90%, or 100% greater than expression of the nucleotide sequence of interest in non-cardiac tissue. By “ventricle-preferred” is intended that expression of the heterologous sequence is most abundant in ventricle tissues, while some expression may occur in other tissue types. Ventricle-preferred expression of a nucleotide sequence of interest occurs at levels at least 1%, 5%, 10%, 20%, 30%, 40%, 50%, 60%, 70%, 80%, 90%, or 100% greater than expression of the heterologous nucleotide sequence of interest in non-ventricular tissue. In an embodiment, ventricle-preferred expression of a heterologous nucleotide sequence natively expressed in atrial tissue may be desired. For example, ventricular expression of the atrial myosin heavy chain isoform may be desired. Expression of a heterologous nucleotide sequence from a ventricle-preferred promoter may not impact atrial expression of the nucleotide sequence operably linked to its native promoter. By “atria-preferred” is intended that expression of the heterologous sequence is most abundant in atrial tissues, while some expression may occur in other tissue types. Atria-preferred expression of a heterologous nucleotide sequence occurs at levels at least 1%, 5%, 10%, 20%, 30%, 40%, 50%, 60%, 70%, 80%, 90%, or 100% greater than expression of the heterologous nucleotide sequence in non-atrial tissue.

By “heterologous nucleotide sequence” is intended a sequence that is not naturally occurring with the promoter sequence. While this nucleotide sequence is heterologous to the promoter sequence, it may be homologous, or native, or heterologous, or foreign, to the animal host.

It is recognized that the promoters may be used with their native coding sequences to increase or decrease expression resulting in a change in phenotype in the cardiac tissue of the transformed animal.

The isolated promoter sequences of the present invention can be modified to provide for a range of expression levels of the heterologous nucleotide sequence. Thus, less than the entire promoter regions may be utilized and the ability to drive cardiac-preferred expression retained. However, it is recognized that expression levels of mRNA may be altered and usually decreased with deletions of portions of the promoter sequences. Generally, at least about 20 nucleotides of an isolated promoter sequence will be used to drive expression of a nucleotide sequence.

It is recognized that to increase transcription levels or to alter tissue specificity, enhancers and/or tissue-preference elements may be utilized in combination with the promoter regions of the invention. For example, quantitative or tissue specificity upstream elements from other cardiac-preferred promoters may be combined with the promoter regions of the invention to augment cardiac-preferred transcription. Such elements have been characterized, for example, the murine TIMP-4 promoter (Rahkonen, et al. (2002) Biochim Biophys Acta 1577:45-52), A and B-type natriuretic peptide promoters (Grepin et al. (1994) Mol. Cell Biol. 14:3115-29), human cardiac troponin I promoter (Dellow, et al. (2001) Cardiovasc. Res.50:3-6), mouse S100A1 promoter (Kiewitz, et al. (2000) Biochim Biophys Acta 1498:207-19), salmon cardiac peptide promoter (Majalahti-Palviainen, et al (2000) Endocrinology 141:731-740), GATA response element (Charron et al. (1999) Molecular & Cellular Biology 19:4355-4365) and the like, herein incorporated by reference.

Other enhancers are known in the art that would alter the tissue specificity by driving expression in other tissues in addition to cardiac tissue, such as in skeletal tissue, CNS tissue, pulmonary tissue, salivary tissue, lacrimal tissue, and vascular tissue, among others. These include, for example, upstream elements from the promoter of the aquaporin-5 promoter (Borok, et al. (2000) J. Biol. Chem. 275:26507-14, herein incorporated by reference) which would give pulmonary and salivary-preferred expression in addition to cardiac-preferred expression. Another example includes upstream elements from the human alpha-skeletal actin promoter, which would give expression in skeletal muscle, in addition to cardiac-preferred expression.

Modifications of the isolated promoter sequences of the present invention can provide for a range of expression of the heterologous nucleotide sequence. Thus, they may be modified to be weak promoters or strong promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts; conversely, a strong promoter drives expression of a coding sequence at a high level, or at about 1/10 transcripts to about 1/100 transcripts to about 1/1000 transcripts.

The nucleotide sequences for the cardiac-preferred promoter disclosed in the present invention, as well as variants and fragments thereof, are useful in the genetic manipulation of any animal when operably linked with a heterologous nucleotide sequence whose expression is to be controlled to achieve a desired phenotypic response. By “operably linked” is intended the transcription of the heterologous nucleotide sequence is under the influence of the promoter sequence. In this manner, the nucleotide sequences for the promoters of the invention may be provided in expression cassettes along with heterologous nucleotide sequences for expression in the animal of interest, more particularly in the heart of the animal.

Such expression cassettes will comprise a transcriptional initiation region comprising one of the promoter nucleotide sequences of the present invention, or variants or fragments thereof, operably linked to the heterologous nucleotide sequence whose expression is to be controlled by the cardiac-preferred promoters disclosed herein. Such an expression cassette is provided with at least one restriction site for insertion of the nucleotide sequence to be under the transcriptional regulation of the regulatory regions. The expression cassette may additionally contain selectable marker genes.

The expression cassette will include in the 5′-to-3′ direction of transcription, a transcriptional and translational initiation region, and a heterologous nucleotide sequence of interest. In addition to containing sites for transcription initiation and control, expression cassettes can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.).

The expression cassette comprising the promoter sequence of the present invention operably linked to a heterologous nucleotide sequence may also contain at least one additional nucleotide sequence for a gene to be co-transformed into the organism. Alternatively, the additional sequence(s) can be provided on another expression cassette.

The regulatory sequences to which the polynucleotides described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

Where appropriate, the heterologous nucleotide sequence whose expression is to be under the control of the promoter sequence of the present invention and any additional nucleotide sequence(s) may be optimized for increased expression in the transformed animal. That is, these nucleotide sequences can be synthesized using species preferred codons for improved expression, such as rabbit-preferred codons for improved expression in rabbits or mouse-preferred codons in mice. Methods are available in the art for synthesizing species-preferred nucleotide sequences. See, for example, Wada et al. (1992) Nucleic Acids Res. 20 (Suppl.), 2111-2118; Butkus et al. (1998) Clin Exp Pharmacol Physiol Suppl. 25:S28-33; and Sambrook et al. (1989) Molecular Cloning: A Laboratory Manual 2nd. ed., Cold Spring Harbor Laboratory Press, Cold Spring Harbor, N.Y., herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Nat. Acad. Sci. USA 86:6126-6130); potyvirus leaders, for example, TEV leader (Tobacco Etch Virus) (Allison et al. (1986)); MDMV leader (Maize Dwarf Mosaic Virus) (Virology 154:9-20); and human immunoglobulin heavy-chain binding protein (BiP) (Macejak et al. (1991) Nature 353:90-94). Other methods known to enhance translation and/or mRNA stability can also be utilized, for example, introns, and the like.

In those instances where it is desirable to have the expressed product of the heterologous nucleotide sequence directed to a particular organelle, particularly the mitochondria, the nucleus, the endoplasmic reticulum, the Golgi apparatus; or secreted at the cell's surface or extracellularly; the expression cassette may further comprise a coding sequence for a transit peptide. Such transit peptides are well known in the art and include, but are not limited to, the transit peptide for the acyl carrier protein, the small subunit of RUBISCO, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose; in vitro mutagenesis; primer repair; restriction; annealing; substitutions, for example, transitions and transversions; or any combination thereof may be involved.

Reporter genes or selectable marker genes may be included in the expression cassettes. Examples of suitable reporter genes known in the art can be found in, for example, Ausubel et al. (2002) Current Protocols in Molecular Biology. John Wiley & Sons, New York, N.Y., herein incorporated by reference.

Selectable marker genes for selection of transformed cells or tissues can include genes that confer antibiotic resistance. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sulfonamide (Guerineau et al. (1990) Plant Mol. Biol. 15:127-136); puromycin (Abbate et al (2001) Biotechniques 31:336-40; cytosine arabinoside (Eliopoulos et al. (2002) Gene Ther. 9:452-462); 6-thioguanine (Tucker et al. (1997) Nucleic Acid Research 25:3745-46).

Other genes that could serve utility in the recovery of transgenic events but might not be required in the final product would include, but are not limited to, examples such as GUS (b-glucoronidase; Jefferson (1987) Plant Mol. Biol. Rep. 5:387); GFP (green fluorescence protein; Wang et al. (2001) Anim Biotechnol 12:101-110; Chalfie et al. (1994) Science 263:802), BFP (blue fluorescence protein; Yang et al. (1998) J. Biol. Chem. 273:8212-6), CAT; and luciferase (Riggs et al. (1987) Nucleic Acid Res. 15 (19):81 15; Luchrsen et al. (1992) Methods Enzymol. 216: 397-414).

Any of the regulatory or other sequences useful in expression vectors can form part of the transgenic sequence. This includes intronic sequences and polyadenylation signals, if not already included.

In one embodiment, the animal cell can be a fertilized oocyte or embryonic stem cell that can be used to produce a transgenic animal comprising at least one stably transformed expression cassette comprising the heterologous nucleotide sequence. Alternatively, the host cell can be a stem cell or other early tissue precursor that gives rise to a specific subset of cells and can be used to produce transgenic tissues in an animal. See also Thomas et al., (1987) Cell 51:503 for a description of homologous recombination vectors. The vector is introduced into an embryonic stem cell line (e.g., by electroporation) and cells in which the introduced gene has recombined with the genome are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the recombined DNA in their germ cells can be used to breed animals in which all cells of the animal contain the recombined DNA by germ line transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rabbit, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a cardiac component and identifying and evaluating modulators of cardiopathic phenotypes.

Methods for generating transgenic animals via embryo manipulation and microinjection, particularly animals such as mice, have become conventional in the art and are described, for example, in U.S. Pat. Nos. 4,736,866; 4,870,009; 4,873,191; and in Hogan, B., Manipulating the Mouse Embryo, (Cold Spring Harbor Laboratory Press, Cold Spring Harbor, N.Y., 1986).

Similar methods are used for production of other transgenic animals. A transgenic animal can be produced by introducing nucleic acid into the male pronuclei of a fertilized oocyte, e.g., by microinjection or retroviral infection, and allowing the oocyte to develop in a pseudopregnant female foster animal. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein. Methods for providing transgenic rabbits are described in Marian et al. (1999) J. Clin. Invest. 104:1683-1692 and James et al. (2000) Circulation 101:1715-1721, herein incorporated by reference in their entirety.

Other examples of transgenic animals include non-human primates, sheep, dogs, pigs, cows, goats, mice, and rats.

In another embodiment, transgenic non-human animals can be produced which contain selected systems, which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355. If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female foster animal will be a clone of the animal from which the cell, e.g., the somatic cell, is isolated.

In an embodiment, the invention provides a method of altering expression of a heterologous nucleotide sequence in an animal, particularly of altering cardiac-preferred expression of the heterologous nucleotide sequence. In the method the heterologous nucleotide sequence is operably linked to a cardiac preferred promoter such as the nucleotide sequence presented in SEQ ID NO:1 or SEQ ID NO:2. An expression cassette comprising the cardiac preferred promoter operably linked to the heterologous nucleotide sequence is used to transform an animal. Animal transformation methods are known in the art and reviewed elsewhere herein. The method yields a stably transformed transgenic animal exhibiting altered expression of a heterologous nucleotide sequence.

By “altered cardiac-preferred expression” is intended that the expression of the heterologous nucleotide sequence in a transgenic cell or cardiac tissue of a transgenic animal of the invention differs from expression levels in a non-transgenic cell or cardiac tissue of a non-transgenic animal by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. The difference may be an increase or decrease in expression levels.

Methods of determining expression levels are known in the art and include, but are not limited to, qualitative Western blot analysis, immunoprecipitation, radiological assays, polypeptide purification, spectrophotometric analysis, Coomassie staining of acrylamide gels, ELISAs, RT-PCR, 2-D gel electrophoresis, microarray analysis, in situ hybridization, chemiluminescence, silver staining, enzymatic assays, ponceau S staining, multiplex RT-PCR, immunohistochemical assays, radioimmunoassay, colorimetric analysis, immunoradiometric assays, Northern blotting, fluorometric assays and SAGE. See, for example, Ausubel et al, eds. (2002) Current Protocols in Molecular Biology, Wiley-Interscience, New York, N.Y. and Coligan et al (2002) Current Protocols in Protein Science, Wiley-Interscience, New York, N.Y., herein incorporated by reference. Analysis of myosin isoform expression is described elsewhere herein.

Transgenic animals that exhibit altered cardiac preferred expression of the heterologous nucleotide sequence are useful to conduct assays that identify compounds that affect cardiac function. The altered cardiac-preferred expression of the heterologous nucleotide sequence may result in altered susceptibility to a cardiopathy.

A “cardiopathy” is any disorder or condition involving the heart or cardiac tissue. Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy; hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; Brock's disease, neoplastic heart disease, including but not limited to, primary cardiac tumors, such as myxoma, lipoma, papillary fibroelastoma, rhabdomyoma, and sarcoma, and cardiac effects of noncardiac neoplasms, congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia; disorders involving cardiac transplantation; myocardial stunning; arterial hypertension; peripartum cardiomyopathy; alcoholic cardiomyopathy; supraventricular tachycardia, bradycardia; atrial flutter; hydrops fetalis; extrasystolic arrhythmia; fetal cardiac arrhythmia; endocarditis; atrial fibrillation; idiopathic dilated cardiomyopathy; Chagas' heart disease; long QT syndrome; and Brugada syndrome.

A “cardiomyopathy” is any disorder or condition involving cardiac muscle tissue. Disorders involving cardiac muscle tissue include, but are not limited to, myocardial disease, including but not limited to dilated cardiomyopathy, hypertrophic cardiomyopathy, restrictive cardiomyopathy, myocardial stunning, and myocarditis; rheumatic fever; rhabdomyoma; sarcoma; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia; disorders involving cardiac transplantation; arterial hypertension; peripartum cardiomyopathy; alcoholic cardiomyopathy; supraventricular tachycardia; bradycardia; atrial flutter; hydrops fetalis; extrasystolic arrhythmia; fetal cardiac arrhythmia; endocarditis; atrial fibrillation; idiopathic dilated cardiomyopathy; Chagas' heart disease; long QT syndrome; and Brugada syndrome.

By “altered susceptibility” is intended that a transgenic animal of the invention differs from a non-transgenic animal in the extent to which the transgenic animal of the invention exhibits a cardiopathic phenotype. The cardiopathic phenotype may present during any stage of development including, but not limited to, embryonically, post-natally, in the adult, and as the animal nears end of lifespan. In an embodiment, the cardiopathic phenotype may be induced by external stimuli such as, but not limited to, diet, exercise, chemical treatment, or surgical procedure. In an embodiment, a transgenic rabbit of the invention exhibits decreased susceptibility to cardiopathy. In an embodiment, a transgenic rabbit of the invention exhibits increased susceptibility to cardiopathy.

Cardiopathic phenotypes include, but are not limited to, hypertrophy; morphology, such as interventricular septal hypertrophy; left ventricular-end systolic dP/dtₘₐₓ or end-diastolic dimension(r); papillary muscle dimension; left-ventricular outflow tract obstruction; midventricular hypertrophy; apical hypertrophy; asymmetrical hypertrophy; concentric enlarged ventricular mass; eccentric enlarged ventricular mass; sarcomere structure; myofibril function; receptor expression; heart rate; ventricular systolic pressure; ventricular diastolic pressure; aortic systolic pressure; aortic diastolic pressure; contractility; interstitial fibrosis; cardiomyocyte disarray; Ca²⁺ sensitivity; Ca²⁺ release; Ca²⁺ uptake; catecholine sensitivity; α-adrenergic sensitivity; beta-adrenergic sensitivity; dobutamine sensitivity; thyroxine sensitivity; angiotensin-converting enzyme inhibitor sensitivity; amiodarone sensitivity; lidocaine sensitivity; glycoprotein receptor antagonist sensitivity; anabolic steroid sensitivity; carnitine transport irregularities; left ventricular dilation, reduced left ventricular ejection fraction; left atrial dilatation; diuretic sensitivity; volemia; ischemia; leukocyte flow properties; the polymorphonuclear leukocyte (PMN) membrane fluidity; PMN cytosolic Ca²⁺ content; high interventricular septal defects, rosette inhibition effect; contractile force transmission; myocardial fiber disarray; increased chamber stiffness; impaired relaxation; small-vessel disease; dyspnea; angina; presyncope; tachycardia; syncope; and the like. See, for example, Braunwald et al. (2002) Circulation 106:1312-1316 and Wigle et al. (1995) Circulation 92:1680-1692, hereby incorporated by reference in their entirety.

Methods for measuring cardiopathic phenotypes are known in the art and include, but are not limited to, echocardiography, transesophageal echocardiography, exercise tests, urine/catecholamine analysis, EIAs, light microscopy, heart catheterization, dynamic electrocardiography, MRI, multiplex RT-PCR, positron emission tomography, angiography, magnetic resonance spin echo, short-axis MRI scanning, Doppler velocity recordings, Doppler color flow imaging, stress thallium studies, cardiac ultrasound, chest X-ray, oxygen consumption test, electrophysiological studies, auscultation, scanning EM, gravimetric analysis, Holter monitoring, hematoxylin and eosin staining, trichrome staining, 2-D echocardiography, cardiotocography, baseline M-mode echocardiography, and myocardial lactate production assays. See, for example, Braunwald et al. (2002) Circulation 106:1312-1316; Sohal et al. (2001) Circulation Res. 89:20-25; Nagueh et al. (2000) Circulation 102:1346-1350; and Wigle et al. (1995) Circulation 92:1680-1692, hereby incorporated by reference in their entirety.

In an embodiment, a transgenic animal of the invention may be used to identify anti-cardiopathic compounds. An “anticardiopathic” compound modulates a cardiopathic phenotype. Modulation may be an increase or decrease in a cardiopathic phenotype. An anticardiopathic compound will modulate a cardiopathic phenotype by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. Methods for assaying cardiopathic phenotypes are described elsewhere herein. Any method of assaying a cardiopathic phenotype known in the art may be used to monitor the effects of the compound of interest on a transgenic animal of the invention.

To identify anti-cardiopathic compounds, multiple transgenic animals of the invention, e.g. at least a first and second transgenic animal, are provided. The terms “first,” “experimental,” or “test” transgenic animal refer to a transgenic animal to which a compound of interest is administered. The terms “second” or “control” transgenic animal refer to a transgenic animal to which a placebo is administered. In an embodiment, the first and second transgenic animals are clonal, age-matched, gender-matched, and subject to similar environmental conditions. In an embodiment, more than one animal may be a first transgenic animal. In an embodiment more than one animal may be a second transgenic animal.

After administration of either the compound of interest or the placebo, the first and second transgenic animals are incubated for a period of time. The period of time will have a predetermined duration appropriate to analysis of the cardiopathic phenotype. Such durations include, but are not limited to, 30 seconds; 1, 5, 10, 30, or 60 minutes; 8, 12, 24, 36, or 48 hours;3, 4, 5, 6, or 7 days; 2, 3, or 4 weeks; 2, 3, 4, 5, 6, 7, 8, 9, 10, 11, or 12 months; up to 3 years. Monitoring of a cardiopathic phenotype may occur continuously; at a single interval; or at multiple intervals, such as, but not limited to, hourly, daily, weekly, and monthly. Any method of assaying a cardiopathic phenotype known in the art may be used to monitor the effects of the compound of interest on a transgenic animal of the invention.

The term “administer” is used in its broadest sense and includes any method of introducing a compound into a transgenic animal of the present invention. This includes producing polypeptides or polynucleotides in vivo as by transcription or translation in vivo of polynucleotides that have been exogenously introduced into a subject. Thus, polypeptides or nucleic acids produced in the subject from the exogenous compositions are encompassed in the term “administer.”

A “compound” comprises, but is not limited to, nucleic acid molecules, peptides, peptidomimetics, lipids, antibodies, receptor inhibitors, ligands, sterols, steroids, hormones, kinases, kinase inhibitors, agonists, antagonists, ion-channel modulators, diuretics, enzymes, enzyme inhibitors, carbohydrates, deaminases, deaminase inhibitors, G-proteins, G-protein receptor inhibitors, ACE inhibitors, hormone receptor modulators, alcohols, reverse transcriptase inhibitors, neurotransmitter inhibitors, angiotensin converting enzyme inhibitors, digitalis, neurotransmitter receptor modulators, negative inotropic agents, β-blockers, Ca²⁺ antagonists, disopyramide, anti-arrhythmia agents, flecainide, and vasodilators. A compound may additionally comprise a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a carboxypeptidase protein or anti-carboxypeptidase antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Anti-cardiopathic compounds identified by the methods of this invention may be used in the treatment of human individuals.

The following examples are offered by way of illustration and not by way of limitation.

### EXPERIMENTAL

### Example 1

### Generation of Transgenic Rabbits

On day 1 a New Zealand White doe was superovulated with 150 U pregnant mare serum gonadotropin delivered subcutaneously under the scruff of the neck. On day 3, the donor doe and a recipient doe received 150 U of human choriogonadotropin administered into an ear vein. The donor doe and a nontransgenic buck were mated. On day 4, the eggs were harvested from the donor doe. To generate the transgenic α-myosin heavy chain ventricle-preferred expressing rabbits, the α-myosin heavy chain nucleotide sequence was operably linked to the β-myosin heavy chain promoter to yield the sequence set forth in SEQ ID NO:5.

Purified DNA comprising the desired transgene was injected into the pronuclei of viable eggs. The viable eggs were transplanted into the fallopian tube of the pseudopregnant recipient doe. The recipient doe was moved to a nesting cage 2 to 3 days before the expected delivery date.

Transgenic offspring were identified by PCR and genomic Southern analysis using ³²P-labelled oligonucleotide probes corresponding to the human growth hormone sequences that were incorporated into the transgenic construct. The founder rabbits were up to 5 months (females) or 6 months (males) before a breeding program was established. Diploid copy number was determined with DNA dot blots.

### Example 2

### Cardiac Preferred Expression from the β-Myosin Heavy Chain Promoter

Expression of the CAT reporter gene under the control of the β-myosin heavy chain promoter (SEQ ID NO:2) was analyzed by ELISA. Transgenic rabbits comprising the β-myosin heavy chain promoter operably linked to the CAT reporter gene were prepared using the method described elsewhere herein. Four stable transgenic lines were established. The genomes of lines 415, 428L, 428M, and 428H contained two, fourteen, twenty-four, or thirty-nine copies of the transgene, respectively.

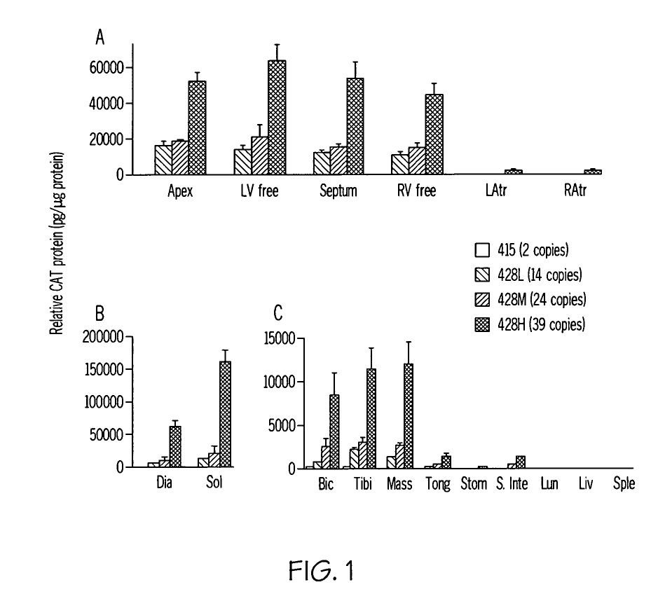

Tissues were dissected from multiple animals in the F2 generation of each transgenic line. Apex, left ventricle, right ventricle, left atria, right atria, diaphragm, soleus, bicep muscle, tibialis muscle, masseter muscle, tongue, stomach, small intestine, lung, liver, and spleen tissues were analyzed. The dissected tissues were frozen in liquid nitrogen. Proteins were isolated by tissue homogenization in 200-400 μl 0.25M Tris pH 7.8 with a Tekmar homogenizer (Tekmar Co). The homogenate was incubated at 65° C. for 10 minutes, then centrifuged for 10 minutes at 12,000 rpm in a tabletop microfuge. The supernatant was transferred to a new tube. The total protein concentration was determined by the Bradford method. The ELISAs were performed with a microtiter kit according to the manufacturer's instructions (Boehringer-Mannheim). The results are reported in pg CAT/μg total protein (FIG. 1).

### Example 3

### Assessment of Myosin Isoform Expression

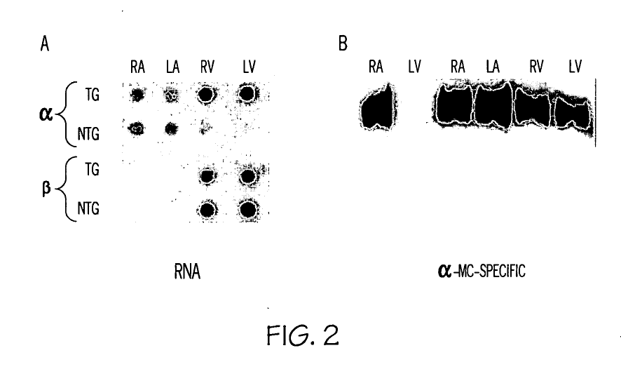

To analyze transgene expression, mice were sacrificed by CO₂ asphyxiation. RNA was extracted using TriReagent® (Molecular Research, Inc.) according to the manufacturer's protocols. Two-fold serial dilutions of the RNAs starting with 8 micrograms were applied to nitrocellulose paper. Transcript-specific oligonucleotide probes for alphα-myosin heavy chain and glyceraldehyde phosphate dehydrogenase (so that any minor variations in loading could be accounted for) were end-labeled with ³²P-ATP and hybridized under standard conditions for 5 hours. Transcript levels were corrected for background and normalized to GAPDH signal intensity. All hybridization signals were quantified on a STORM® Phosphor Imager (Molecular Dynamics). The results from one such experiment are presented in FIG. 2, Panel A.

Total or myofibril protein samples were loaded onto a 7.5% SDS-PAGE gel and resolved by electrophoreses at 120 volts for 2 hours. Proteins were transferred onto nitrocellulose at 4° C. overnight. Western analyses were performed using an alpha myosin-specific antibody. The results from one such experiment are presented in FIG. 2, Panel B.

### Example 4

### Echocardiographic Analysis of Transgenic Rabbits

Transgenic rabbits from the F1 generation, prepared as described above, were aged 2-12 months before physiological analysis by echocardiography and/or 5-15 months for cardiac catheterization. Each rabbit was sedated with ketamine and the chest was shaved. Echocardiography was performed using a Hewlett-Packard 5500 Ultrasound System and a 7.5 MHz transducer. Contrast echocardiography was performed using intravenous Optison (Mallinckrodt). The 2-D and M-mode images were recorded on videotape and analyzed off-line by methods known to one of skill in the art. (See, for example, Schiller et al. (1989) J. Am. Soc. Echocardiogr. 2:358-367, herein incorporated by reference.)

### Example 5

### Cardiac Catheterization of Transgenic Rabbits

Transgenic rabbits of the invention were initially sedated with ketamine then anesthetized with isoflurane. Femoral access was obtained via cutdown and a 4 Fr sheath (Cook) was placed in the artery. A 4 Fr pigtail (Cook) was advanced into the sheath and positioned in the left ventricle. Pressure measurements and the electrocardiogram were recorded with a Prucka Cardio Lab 4.11 physiologic monitoring system (GE-Marquette). Contrast angiography was performed with Optiray 350 (Mallinckrodt) contrast diluted 1:1 with normal saline using a Liebel-Flarsheim Angiomat Illumena Digital Injection System (Mallinckrodt). Cineangiography was recorded with an OEC Series 9800 Digital Cardiac Imaging System (General Electric) in the left axial oblique and right axial oblique projections.

Following the procedure, the catheters were withdrawn, the femoral artery ligated, and the incision closed. The rabbits recovered from anesthesia before being returned to their cages.

### Example 6

### Assessment of Myosin Isoform Activity

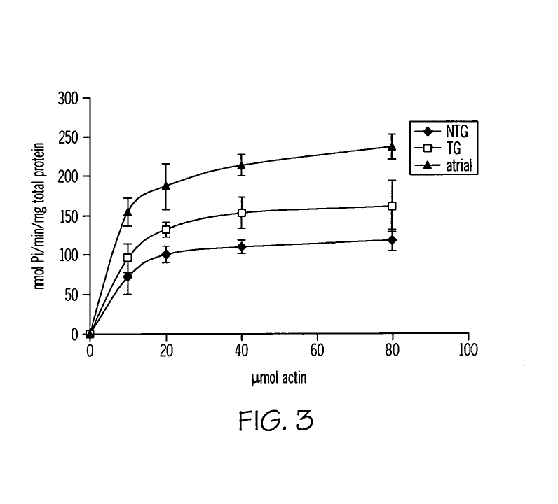

Ventricles and atria were isolated from transgenic and nontransgenic rabbits. Cardiomyocytes were obtained from the ventricular and atrial tissue. The cardiomyocytes were lysed and used in ATPase assays. Reaction mixtures comprising cardiomyocyte lysates, ³²P-ATP, and salts were prepared. The assays were initiated with 10, 20, 40, or 80 μmol actin. The reactions were quenched. Reaction products were resolved and the nmol of inorganic phosphate (Pi) per minute per milligram of total protein in the reaction was determined. Results of such an experiment are presented in FIG. 3. Non-transgenic atrial tissue contains primarily the α-myosin heavy chain isoform. Non-transgenic ventricle tissue contains primarily the β-myosin heavy chain isoform. Transgenic ventricle tissue contains both the α and β myosin heavy chain isoforms.

### Example 7

### Induction of Cardiomyopathy by Heart Rate Elevation

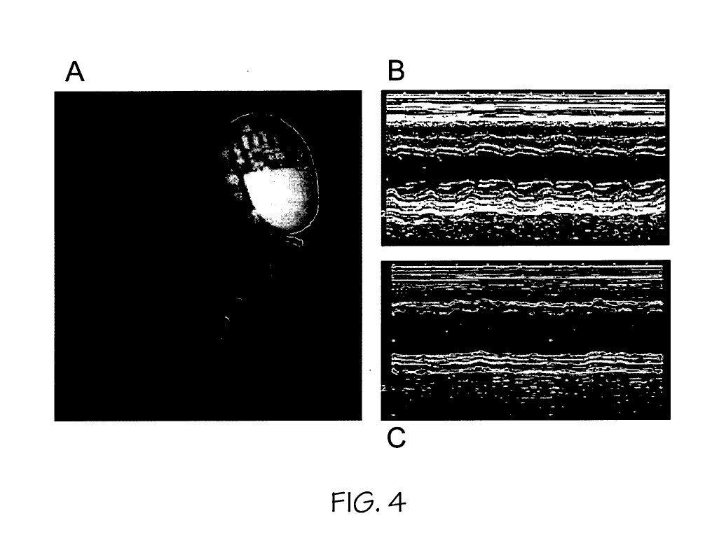

Pacemakers were modified and implanted in twelve age-matched rabbits (5 transgenic and 7 non-transgenic littermates), as shown in FIG. 4. Five to seven days after surgical implantation of the pacemakers, rapid VVI pacing was initiated at 300 beats per minute (bpm) for 10 days. The pacing rate was then increased to 340 bpm for 10 days, followed by 10 days at 380 bpm. All 5 transgenic animals survived the pacing protocol. One non-transgenic rabbit died with a noticeably dilated heart after the pacemaker rate was increased to 380 bpm. The surviving animals did not exhibit signs of overt distress consistent with congestive heart failure. M-mode echocardiograms were performed on the animals. M-modes of a transgenic (FIG. 4, Panel B) and non-transgenic (FIG. 4, Panel C) rabbit are indicated.

### Example 8

### Assessment of Response to Cardiomyopathic Stimuli

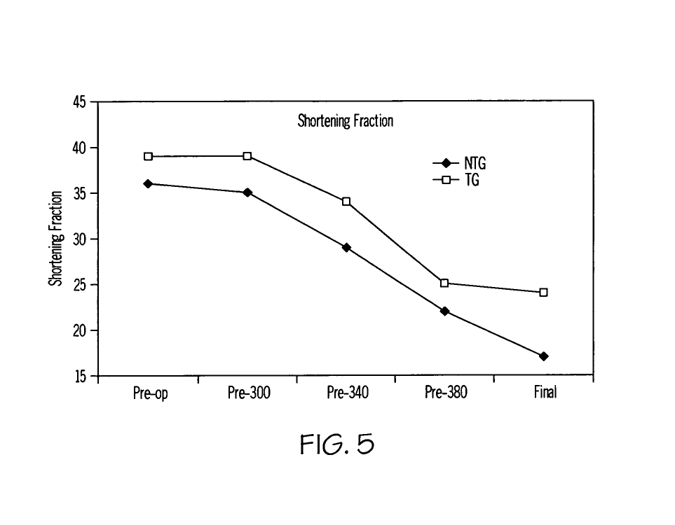

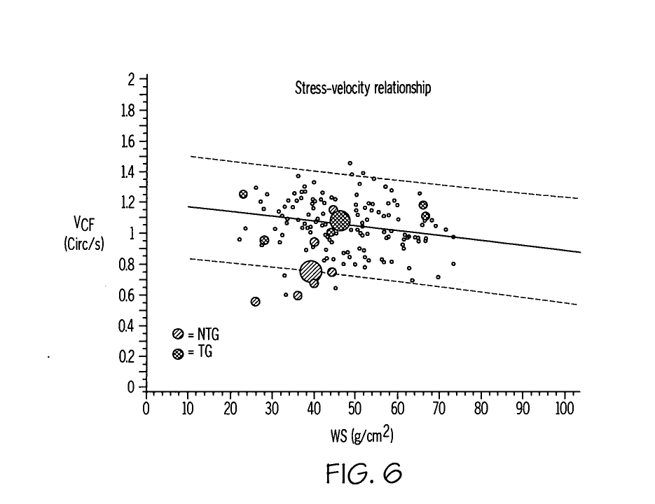

The shortening fractions of the transgenic and non-transgenic rabbits undergoing the previously described cardiac pacing protocol were determined. Shortening fractions were determined prior to implantation of the modified pacemaker and prior to each increase in cardiac rate. M-mode and Doppler echocardiography were performed as described elsewhere herein to assess left ventricular (LV) function and dimensions. Left ventricular percent fractional shortening, velocity of circumferential shortening, and left ventricular end systolic meridional wall stress were calculated. LV mass was calculated using M-mode LV measurements according to American Society of Echocardiography conventions and the modified American Society of Echocardiography-cube LV mass equation. Results from one such experiment are presented in FIGS. 5 and 6.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

Although the foregoing invention has been described in some detail by way of illustration and example for purposes of clarity of understanding, it will be obvious that certain changes and modifications may be practiced within the scope of the appended claims.

That which is claimed:

1. An animal cell stably transformed with an expression cassette comprising:
a.) a promoter, wherein said promoter comprises a nucleotide sequence selected from the group consisting of:
i.) a nucleotide sequence set forth in SEQ ID NO:1 or SEQ ID No:2;
ii.) a nucleotide sequence having at least 90% identity to a nucleotide sequence set forth in SEQ ID NO: 1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO: 1 or SEQ ID NO:2, wherein said nucleotide sequence is capable of initiating transcription in an animal cell; and
b.) a heterologous nucleotide sequence operably linked to said promoter.

2. The animal cell of claim 1, wherein said animal cell is from a mammal.

3. The animal cell of claim 2, wherein said animal cell is selected from the group consisting of rabbit, mouse, monkey, dog, pig, goat, and cow.

4. The animal cell of claim 1, wherein said animal cell is from cardiac tissue.

5. The animal cell of claim 4, wherein said animal cell is selected from the group consisting of ventricular and atrial tissue.

6. The animal cell of claim 1, wherein said promoter is capable of initiating tissue-preferred transcription.

7. The animal cell of claim 1, wherein said tissue-preferred transcription is cardiac-preferred transcription.

8. The animal cell of claim 7, wherein said cardiac-preferred transcription is ventricle-preferred transcription.

9. The animal cell of claim 7, wherein said cardiac-preferred transcription is atria-preferred transcription.

10. A transgenic rabbit comprising in its genome at least one stably incorporated expression cassette comprising:
a.) a promoter, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO:1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell; and
b.) a heterologous nucleotide sequence operably linked to said promoter.

11. The rabbit of claim 10, wherein said promoter is capable of initiating tissue-preferred transcription.

12. The rabbit of claim 11, wherein said tissue-preferred transcription is cardiac-preferred transcription.

13. The rabbit of claim 12, wherein said cardiac-preferred transcription is ventricle-preferred transcription.

14. The rabbit of claim 12, wherein said cardiac-preferred transcription is atria-preferred transcription.

15. The rabbit of claim 10, wherein said rabbit exhibits altered expression of the heterologous nucleotide sequence.

16. The rabbit of claim 10, wherein said expression is cardiac-preferred expression.

17. The rabbit of claim 10, wherein said heterologous nucleotide sequence comprises a nucleotide sequence selected from the group consisting of:
a.) a nucleotide sequence set forth in SEQ ID NO:3;
b.) a nucleotide sequence having at least 95% identity to the nucleotide sequence set forth in SEQ ID NO:3;
c.) a nucleotide sequence encoding a polypeptide having the amino acid sequence set forth in SEQ ID NO:4; and
d.) a nucleotide sequence encoding a polypeptide having at least 95% identity to the amino acid sequence set forth in SEQ ID NO:4.

18. The rabbit of claim 17, wherein said promoter is capable of initiating ventricle-preferred transcription and said rabbit exhibits altered myosin isoform expression.

19. A transgenic animal comprising in its genome at least one stably incorporated expression cassette comprising:
a.) a promoter, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO:1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell; and
b.) a heterologous nucleotide sequence operably linked to said promoter.

20. The animal of claim 19, wherein said promoter is capable of initiating tissue-preferred transcription.

21. The animal of claim 20, wherein said tissue-preferred transcription is cardiac-preferred transcription.

22. The animal of claim 21, wherein said cardiac-preferred transcription is ventricle-preferred transcription.

23. The animal of claim 21, wherein said cardiac-preferred transcription is atria-preferred transcription.

24. The animal of claim 19, wherein said animal exhibits altered expression of the heterologous nucleotide sequence.

25. The animal of claim 19, wherein said expression is cardiac-preferred expression.

26. The animal of claim 19, wherein said animal is selected from the group consisting of rabbit, mouse, dog, pig, goat, cow, monkey, chimpanzee, and sheep.

27. A method of altering expression of a heterologous nucleotide sequence in an animal, said method comprising:
a.) providing a transgenic animal comprising in its genome at least one stably incorporated expression cassette comprising:
i.) a promoter, wherein said promoter comprises a nucleotide sequence selected from the group consisting of:
a.) a nucleotide sequence set forth in SEQ ID NO:1 or SEQ ID NO:2;
b.) a nucleotide sequence having at least 90% identity to a nucleotide sequence set forth in SEQ ID NO:1 or SEQ ID NO:2, wherein said nucleotide sequence is capable of initiating transcription in a cell of said animal; and
c.) a nucleotide sequence comprising at least 50 contiguous nucleotides of a nucleotide sequence set forth in SEQ ID NO:1 or SEQ ID NO:2, wherein said nucleotide sequence is capable of initiating transcription in a cell of said animal; and
ii.) a heterologous nucleotide sequence operably linked to said promoter; and
b.) determining expression levels of said heterologous nucleotide sequence in said animal.

28. The method of claim 27, wherein said expression is cardiac-preferred expression.

29. The method of claim 27, wherein said animal is selected from the group consisting of rabbit, mouse, dog, pig, goat, cow, chimpanzee, and sheep.

30. The method of claim 27, wherein said expression occurs in cardiac tissue.

31. The method of claim 30, wherein said cardiac tissue is selected from the group consisting of ventricle tissue and atria tissue.

32. The method of claim 27, wherein said expression alters the animal's susceptibility to cardiopathy.

33. The method of claim 32, wherein said cardiopathy is a cardiomyopathy.

34. A method of identifying anti-cardiopathic compounds, comprising the steps of:
a.) providing a first and second transgenic rabbit whose genomes comprise an expression cassette comprising:
i.) a promoter capable of initiating cardiac-preferred transcription, wherein said promoter comprises a nucleotide sequence selected from the group consisting of:
a.) a nucleotide sequence set forth in SEQ ID NO:1 or SEQ ID NO:2;
b.) a nucleotide sequence having at least 90% identity to a nucleotide sequence set forth in SEQ ID NO:1 or SEQ ID NO:2, wherein said nucleotide sequence is capable of initiating cardiac-preferred transcription; and
c.) a nucleotide sequence comprising at least 50 contiguous nucleotides of a nucleotide sequence set forth in SEQ ID NO:1 or SEQ ID NO:2, wherein said nucleotide sequence is capable of initiating cardiac-preferred transcription; and
ii.) a heterologous nucleotide sequence operably linked to said promoter;
b.) administering a compound to said first rabbit;
c.) incubating both the first and second rabbits for a period of time; and
d.) monitoring said first rabbit for a modulation of a cardiopathic phenotype in said first rabbit compared to said second rabbit.

35. A transgenic rabbit comprising in its genome at least one stably incorporated expression cassette comprising:
a.) a promoter, wherein said promoter comprises the nucleotide sequence set forth in SEQ ID NO:1; and
b.) a heterologous nucleotide sequence operably linked to said promoter.

36. A transgenic rabbit comprising in its genome at least one stably incorporated expression cassette comprising:
a.) a promoter, wherein said promoter comprises a nucleotide sequence having at least 90% identity to the nucleotide sequence set forth in SEQ ID NO:1, wherein said nucleotide sequence is capable of initiating transcription in an animal cell; and
b.) a heterologous nucleotide sequence operably linked to said promoter.

37. A transgenic rabbit comprising in its genome at least one stably incorporated expression cassette comprising:
a.) a promoter, wherein said promoter comprises the nucleotide sequence set forth in SEQ ID NO:2; and
b.) a heterologous nucleotide sequence operably linked to said promoter.

38. A transgenic rabbit comprising in its genome at least one stably incorporated expression cassette comprising:
a.) a promoter, wherein said promoter comprises a nucleotide sequence having at least 90% identity to the nucleotide sequence set forth in SEQ ID NO:2, wherein said nucleotide sequence is capable of initiating transcription in an animal cell; and
b.) a heterologous nucleotide sequence operably linked to said promoter.

39. A transgenic rabbit comprising in its genome at least one stably incorporated expression cassette comprising the nucleotide sequence set forth in SEQ ID NO:5.

40. A kit comprising a transgenic rabbit comprising in its genome at least one stably incorporated expression cassette comprising:
a.) a promoter, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO: 1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell; and
b.) a heterologous nucleotide sequence operably linked to said promoter.

41. A kit comprising a transgenic rabbit comprising in its genome at least one stably incorporated expression cassette comprising the nucleotide sequence set forth in SEQ ID NO:5.

42. A kit for performing a method of altering expression of a heterologous nucleotide sequence in a rabbit, said kit comprising a transgenic rabbit comprising in its genome at least one stably incorporated expression cassette comprising:
a.) a promoter, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO:1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell; and
b.) a heterologous nucleotide sequence operably linked to said promoter.

43. A kit for performing a method of altering expression of a heterologous nucleotide sequence in an animal, said kit comprising at least one expression cassette comprising a promoter, wherein said promoter comprises a nucleotide sequence selected from the group consisting of:
a.) a nucleotide sequence set forth in SEQ ID NO:1 or SEQ ID NO:2;
b.) a nucleotide sequence having at least 90% identity to a nucleotide sequence set forth in SEQ ID NO:1 or SEQ ID NO:2, wherein said nucleotide sequence is capable of initiating transcription in an animal cell; and
c.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell.

44. A kit for altering an animal's susceptibility to a cardiopathy, said kit comprising a transgenic animal comprising in its genome at least one stably incorporated expression cassette comprising:
a.) a promoter, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO:1 or SEQ ID NO:2, wherein said nucleotide sequence is capable of initiating transcription in an animal cell; and
iii.) a nucleotide sequence comprising at least 50 contiguous nucleotides of a nucleotide sequence set forth in SEQ ID NO:1 or SEQ ID NO:2, wherein said nucleotide sequence is capable of initiating transcription in an animal cell; and
b.) a heterologous nucleotide sequence operably linked to said promoter.

45. The kit of claim 44, further comprising a non-transgenic animal.

46. The kit of claim 44, wherein said animal is a rabbit.

47. The kit of claim 44, further comprising a non-transgenic rabbit.

48. A kit for identifying anti-cardiopathic compounds, said kit comprising a first transgenic rabbit and a second transgenic rabbit whose genomes comprise an expression cassette comprising:
a.) a promoter capable of initiating cardiac-preferred transcription, wherein said promoter comprises a nucleotide sequence selected from the group consisting of:
i.) a nucleotide sequence set forth in SEQ ID NO:1 or SEQ ID NO:2;
ii.) a nucleotide sequence having at least 90% identity to a nucleotide sequence set forth in SEQ ID NO:1 or SEQ ID NO:2, wherein said nucleotide sequence is capable of initiating cardiac-preferred transcription; and
iii.) a nucleotide sequence comprising at least 50 contiguous nucleotides of a nucleotide sequence set forth in SEQ ID NO:1 or SEQ ID NO:2, wherein said nucleotide sequence is capable of initiating cardiac-preferred transcription; and
b.) a heterologous nucleotide sequence operably linked to said promoter.

49. The kit of claim 48, wherein the genomes of said transgenic rabbits comprise at least one stably incorporated expression cassette comprising the nucleotide sequence set forth in SEQ ID NO:5.


SEQUENCE LISTING (5 sequences)

SEQ ID NO: 1 - DNA, 5190 bp, Oryctolagus cuniculus
ggatccacacaactgggcttcaccccaccaggtgccccagctcaactctgtcttctgtcccagcacccgcccaccttcccaaaactaaccgtgctctatttccttcccagagcttgaatgaggaatagcctggtgacgccttgatccgcccagccctgaggacgacgccagtgaagtcccttgtctgggagctcacatagcagcagccccttgggaagaagcagaataaatcagttttcctcgaagctgagatcctgcctccagactcttcttcactgcctgtcagccctgtgggggtgggggtccagtgggtggggccttggagtgggaaaggtgggagaaattggtggagggtgggtctttagggcatgaggggcagctgcaagggccagacctaggagcttcccgggatcctgtagggtctgagccctctggggagcagccatggtcttgtggactctctttacctgctccggcccaacataacacagaggtgacccgtctcctcacagccctctcctggctcagagtttatcagagttcaagatttaggcaaacctgggcctatctaccatggacatccacccccacctcactgataacacccagcgccagggcatggggaggtctggtatgaagggggctgaacagtaccctttctgcaacctctggggtatgacccacgagggcaggtgaggcaaaggtgtcctgagccccagccccaacagtctcagaggacacaagcccttccagatgcagccagtggctccccccgccacagctaggagccgggcaccccgtgcacaccttcctgtcaccctcggctccctccagagcctgcagccctccaagcaggtgtccctccctcctccccacatcttccactgtagcttccattcccaagactttcctcctctggacatgtaggaggatggcattctttggcccatttatggaaagccatctcagacgggcagaagagagcctggggagggtcacatgtgagcaggactctgggcaggagaggaaagacatgtccccagagggctgaagccggggcagcagcagctggggtggggtggaagaaagggcaggttaaccgcccccccccctgcagtgacaactcagccccttggagtggctgccacagctgggctgccaagggtaggactggggcagtgggatgtggagggcagggccttgggagactgtgggactgtcccccacaccccatcccaagaacctgagtggcccctattcagcccccgtctgaagctcgtccaaacctgccacacacaccgacgcacagccctgttcctgctgcacaccctactctgagtggcagccagaacaagggcctaggccaggcaggaggggataccagtggccgagcacagcaggagaggtgaggggaagagaaaggaaacagggaaggaaggagatacccagggcagatgcaaaggacgagcaggcgtggggtgacaagcacacgggaggaagccgccgggaatgaagaggcctcttagacacacaagccctcacacccacctccctgcacgcctctcctcccttctccctccccacatcattccccttcccaggagcaggttcggagatgaaggaagaggtggaggtcagagaggacacacttgtagggaagcccaggggtctgagcaaccacagtgtgcaagacagcaagagagggaggtgctgggagagtggacagcagtgtcaggagaggcctggagagccgggcccaggagaaggagagagaaggagaggcctggccagggagcccttgagagggagcctgggacacaggcacctgcagagcagagggaggtgacagtaggggaaaggacccgagtcaccttcagaggtcaggagagtctggaaagcagaggaaccagggggtggaagggaggtggccaaggagcccggggcaggatgaggaggcagagagctcagaggagacagagccttaagtcagacccagacccaggctcaggcagacccaggagaatccgaggccaagtctgcctttgagaccgggaaccaggcagggggagaggacacagagacagagacatggggaggcaagcaggagaggggcggcaagccctgcctccctgtccccctacctttggccagagtatccgtcccgtgccctcacactccagggcctccagcagaatgcggagctgatttgggtggcagtggagacagcgtggacagcagaggtgtatgggaacgccaggacagaggagggggcccagggaaggaagagggcaaaccaggccagaccgggcccgagcccagagccaggctaaccccttccttctacctccttccctaggagggcaggcactgcctgcccacccctcagccccgccccctaccacctctcccaagtttggtctgctcagggcgcagggttctcccgaggacaaagcctggtctttgtcacctggaccttacccaggctgacctggtattccagccccttatcaggtccccagggggatgggaaccaggcagtgacgcaccaggccacgggataacccccagcttgtgcactggggcctcaagtgacctagagctagagacaggtggcctgcatcccctgagggcaacggtttggcatgtgcacctgcaggagatgggaatggccggtcaactcaggtccctccaaggacactgagtctggggtccaagtcccccagagagagatgtgtctctgagccccgtaataggcagccagcatcctagggggctgacaaccttggggggaaccccatcttcacggcaagtgctcccacccagagaagctgacccctgcctgtcctacccacctccacaccctggagctatatcgagaggtgtcagtggataggggtgggaggggagctgaggcagtattcttgggtgtgagggtgtgggggaaagccagggcaggagagtctggctttgtcttctgaacacaatgtccacttagtcacaacaggcacggcctgttgaagacccgacacctaccgcctctgagaggggacagcctgcaggatggaggaggagtaccttgagggctgggtgcttgaggacccccccccccaaagacaggcatgggagtgtgagctcaggaggggatagcaggcttctatctgggaagggggcagactacagagccccctccccccaaccctgcacccccctgcagccctgtggaacgcagagccagagcccaattggtgtaaatccccaggcctgtctggcgccccagccatcctcggcagcacctgcaccctctggcagcctggagaggcggaagggagcggcccccacccccacccctgctccctcaggcccggggattagtgcagaaggtggcagcaggagggtaaaagcctttgcgtgcagatgtgagcatcggcctgctggaaagcacgcagggatctctgtaagctgaagtgtgaaatctgcatgtgtgtctggtcaagtctgcacgcgagggtgctggaccaagggtctgtgcgcaggtgtgcctgaggcagcgtggagaattccttccactgggtgatgggtttgcatgagggccactgtatggggagcaccggggaagtgctggggctccagatgcattcccaagcaaagcagccttgagtcctctgtcagacacctgcctgggaatagaagcccaggcacagcgtgtgtgcagagttggggcaggacagagagggaaactgagtctggagctggctagtagacgcctgctccagctcacaggtccaggtctgagagtgtcgggagacacttgcagcctgggctgtgtgaagacaggcctgggtaaatgggagagggtaggggacaagggagggaaactgcagctggggggcaggggacaagcattcgtcctatatgaaaaggtgaccctcaccccagtgtgctcaactcacccttcaggttaaaaataaccgaggtaaaggccatggtggggcgggggaggtggtggagaaggtcctgtcttcccactatctgcccatcagcgctctggaggggcggaatgtgctcaaggactaaaaaaaggccctggagcccgaggggctggggcagcagacctttcatgggcaaatctgggggccctgccgtcctcttgtcacctccagagccaagggatcaagggaggaggagccaggcagggagagaggtgggagggggtccctccggaagggctccaaatttaggcaagggggtgggggaagtggcatataaaggagctgcagcgttgagggcagccagactcctgcagcccaggtaagtgtggtttgtgcaggggcactgtgcagctgtgcacagggaccctgccttcctgggagtgagctgcagggcaggcgggggcccacgggttctgctggaagcgcccttctccagccctgtggcaggtccatgagaagcgcgactcctgtctcctgtctctctggttccctgcccggttctcagcacttgctccctcctttgggtggcttcctgcccgttggccctgtgctgctttgcctccctgtcttggtcagcacttgtcccagtcctcaatctccgtctccctccttccttgccctcccttcgtgcctgtgtgttcttttctccctgcctttctctgtttctgtctctgcttccacacccctgccctcccacattcactcactctgcatcccttcctttcttcctactccctgccggtccaccctgctggtgtctgtcggctctctgggctgggcctcctcgtcccatcacttgtctccatgattctcttctctgcactttgtggctcctgctcctccagtggttcagattctccgggattccctgtgaggagagagtcaggtgagcggcacccagtctcctctgcagacagcagagcaggctttctccgggagccaggtccaccccctggcaaccccaggcactccttccccccatgccgacctcttacaaagatcccccctcagccccagctgaatggggtgggtggccccaggcagatcctttcacctgggtaatgagaggagagcaataggtcctggaaaggaggtggtggggcaaggaatgggaatgggaaagacagggtcacccacgaaagcctgagtctgcctcgggagtcacaggaccccctgtcttggccccaggggagaggtcgac

SEQ ID NO: 2 - DNA, 6921 bp, Oryctolagus cuniculus
ggatcccccctcctcggcccccagcttgctcccaggacagaacaacatggatcatgctgtgttgtcctgaaccagtctcccctggttcctggtgtgttgcctttggcacccctggatgaggggaatgcctgtgagtcaggcgccatgagatcccactgcagctgccatagagcctggctacagcaggggtcagtatggcatctggtgggctgaccaatcggaacccctctgcagggagggaggaggatgagacaggctgagctgggggctggaatggtgtcactcctggcaagtccatcagacacccatggtctgtctcagaactggctatggaaggtagcttgggctctgtgcaatctgtttgcacaaagatatatgcatacacatatgtgtctgcaaaggtaggcatgtgtgcttgcgtgcactcaaatgcatttaaggggtagggtagagctgggggtgggggacaggcagtggctctggggaggtctgcttaaggaggtagagcccttaccaaagaaatgaagtactgggggctggcactgtgtggcatagtgggtaaaagctgccacctgccatgctggcaacccatgtgggcgccagttcgagtcctggccgctccacttccgatccagctttctgccatggcctgggaaagcagtggaagatggcccaagtccttgggaccatgcacccacgtgagagacctggaagaagctcctggctcctggctttggatcggcgcagctctggccgttgtggccaatcggggagtgaatcaatgggcggaagacctctctctctctctctctctctctctctctctctctctctctccgcctctccttctctctgtataactctgactttcaaataaataaatcttaaaaaaaaaaaatgaagtcctgtaatccctttcccaggggagaagactgatgatttctcagccacttgggctcctcacaaagcaattaccttcagctctgagtaaatcccacggtctccatatcctgtgctggtccaggtgtggcggccacaggatgctgccccaagaacacctgtggacagagcacaggttctcaggaggagcagagggagcagacatctgctctgtgtgcccagcacagagacagcagtgtgcagggatcctggcttggacacaaatgcatctggcaggtgacccaaggatgctgattttccatttgcccgatgatttcacatcataacaccttggccattctccccacagagtttctagacccctgcagcctacgattgggctggctccagcgcaggtgtggatgtgactgctgaaggagcacgatcgacccagggctggatctgcccctctcctgggggcctggcttagtctgcctccgctctacagcccgatgcccctctcctgggggcctggcttagtctgcctccgctctacagcccgattcccagaccctagggccagccagctctaccagggtgcaccactagggtccctaagaccttcaggcaggacatgaaaaccagttagctggtgagtggccctaggttcccaactggtgtcctggtggatgtcaccattggacctgggccaactcctggtgggccccaagacagcactggcctgtggggagggccgttctgtgggtctcagtcaggcagtggtggccctcctggctaagggctgggtcttggagggcctgcgtaacttgggctcttgacgtgatgtttccactggcagccatgggctgggtacaggagaccataggactgcagaatacctgaccagccccggcccagaccatcccttgagccagatggacttagtgaggctgggggacaaagaactgcccccacccgccaactgcagtctgacttcagtgggttcgtgctcctgagggtgagaggagccgtttggtgagtgcctaccacgtgtcacgtgtgcactacaccctgtacccacacaatggtcacaacaaccctgggaggtaaatggaccatggccacactacagatgacaagactgtctcagagaggccctgtgactaacccagagcacatggccagaaaacggaggggctggctttcaaaacaactgaccgaagctcccaggctgcccctcacatggtcctgaggcctgcagccctggcgtccagactgcccactccccacaaagctcctccaaacactctaagatggggaagctctgtcttcccaaaccactgcaagctaaaccgagccatccctgcaactgtccactgtcgacattgtggagcagggaagcgagtgtggggtggagggccgaggtacaaaagatcttagaggccgcaggaagacaaaagcatgacaagaaggagcagctgtttgtaagggaaacttgagaccggcacaaagcccagctccctaatcccaggctcctgtggagccagggcgggcccccaaggtcagggatcctaggggcaggcagatggcagatcggcctccaggcccgggtggggacccactcccatgttcacgaacaactgggggcccattgtcctttccgggttaagtccccttcctctagtgccccactctgctctcttccctttgggtttcgggggtcccttccagcttccttctttctgtgcggggtcccttttagtgtgccctacttcaccccctccccaattcctgggagtaattttagcaacttttcctctgaactgccgctggcatggtcacagcaaatggcccctgtttgggcctgacactcgatcagggtggtgacagagcccagacaaagcaggcacagagcccggggaggggggacaggagacagatcaccttccttccactccaggccttatttggtctaccttggtgaagctgccctccctccagccccttttatttatagccttttgctctgctctcctttgtcttgtcggctatcactctgcagtgccctctgcacaacccccctcctcccccctcccctccatggggccctggcccctcactctccctctaggaatgtgggtgtgtacacagggcaccagctccaagctcaggcccgaactccacttctctcaactgctgaatggagcttctcacgggaggggtctttctctggtcgctctggctagaacaatgcccagtgcgcagcaggcttccagaagcactcgctgagtgaggaagtctgcaggctgctgtccccagatggcagcaggcagaagcaagagaagcaccgtggagccaagggcaggtgctggaggggctgggacgtcagaccccactgccaccactaccaccacctaagcctggagttaggggagtgagttctattgtgcaagagggcttggagactgaacaccaatgctgatacaccgatgacccagagccttccagacttaaaaggaagcacatgaagcatgatcctttactaggtgcaaaaacagctcctgcaaagggttcaaccgaggctcaagcatcttctgtgtgtcttcattgcctgtacctgctcctgagtcctgggaactcggggaagggggcagcccccttctttgcttaggtaaccagaagggacggacgcaagggcttgctgtccatcaccagtccctgaggcccctgagtcctcggcacctgcagggcctggccaggaccctcagcgtctgcagtcagggctttcactggtgcaggctcagcttcagcagctgcaggcccacgggtcatggtccaccccacccccgcccgtctgcaccctgaacacacagtcgcttgtggttctcagacctcgccttggttaccacgcagcccccacagaagcccctcctggagtttctctccccagctgtgcccaccccaccttcaacatgtacctgatccgcaccccatcccccaggcctgcctctccttccaagctgacagccaatcagatggattaatccgcacgcgcccaagagcctcctgggaagaaggggaggggggtggatttcaaaaccaaaccacgcagcggttgcctggacacgaagccagtcaggcttgcctgcaagagccaggctgtgctctgctcagagcctgcgctccggctcccgctcctccattcccttgccctgcccgcctctcagcctcactgtagaacaccagtcgcatccaggtctgggaagaggtcacaccgctggaggaacagctgtcctggttcagggtctcctagcttagggcagctgtgcgatctcccactcctggggcgggggtcacctagtagctgggggcgcagaattgggaagacaagtcaggcgcggggctgactgtgcatcttagtcctgcgctgactaggaccatccagcactgggccccgagctctgcacacagtatgtcttccctccttcccaggactccctccacaggctgctgctatttccccgccctgattagctgggtgaccttaaacccatacttcccctctccataaaacgaaaagcttggacggataaagacctgtctggctctgggactcagtgatctcaacacatcgcctgctgatccaaggaaatcagggcctgagccagaggccccatgtcttagctccctggtcatcgtccgcgccccttagcgaagcactctccctccagctgcccccctccagcccctggttctgccctgccctccctggagctgagacttagtcccttttccatctcacagttggatgcaggctccaggccaggaaagcagggaaatttcccgttgcagaaacggggccaggtcagccttggaggctgggggcctaaggggcagcagcatgaggggcccacagtgccctgtgggagagccagggcctgcctgcctgaggtcacactggtggtcctggtcagttccctctcccacaggcagtggaatgcgaggagatattttctgctgcacgttgagccaccccgccccctggaactcagaccctgcacaccccatgccataacaatgacgaccacttccaattgtttcctggcccgagggggaggggagctctctgggagggggggccctgggggaaatgcttccagtgacaacagccctttctaaatccggctagggactgggtgcaggtgggggtgggggcgccctgctgccccatatatacaacccctgaggccaggtctggctctcagctctctcctgctctgtgtgtctttccttgatgttctcaggtaggagcggggagaagggggctccaggttaggaaggggctcccccaggaacagcaagcttcatcagggctttgtgcagagtcccaggtccggtcgttgagcacacgtgtgcaggttacacacccgtggatgtgccactgagtgtgacgctcctgtattccctggggaaggtgtctgggtctgagagtgtgggaaccaaatgaatgagtatgtgtgtgtgtgtgtgtgtgtgtgtacaagcatgtgcagtcggcaggtgagggaggggtgtgcctcactgtctgacgcagcgagtattgatgatcgtgcctgagtacgagcagaatcttttgggagtgggggggaatggtgtagaatgattgtacaggaagtgtggacgcttccaagctagtcctgagaaccatctgtccatgcacatggaggggcttgcctgtgcgtcgggagtcacgaccgctgtcgtgtgcgtgccccctgcagaaggcgctcccaggcatgtgggtgtggggcttgggtccttctacactaaagcataggcagccacatgagcctgcgtccccccacccaggcacataaacatgcggctccatagggtaccttagggggcgggttctgtggcccaggaggcggcctctgtgtgtgtgagacgtgtgcaagagcaggcatgcatggtcatgggggaggtgcagggtgctcagctgtgtggtgggtgtgtctaagagaggacacctgtcgtgtccacaaagtctgacaatgaatgggccagaaaacaactggctttggagtggggagagaggggcttgaggctcagctctaccacttaccatttatgctcttgaataagcaaatcctgtcaccttggtttccccgtctgatgcatgggatgagaactgagaggagagtttgtggactgcacagcactgagcaagtgtgaccattagctgtgtcccagatagttcaaagcaagtctcactagaaggctatgagcgtctcccaggagtgcggaaacctgttagaactggtccttggggcagcagggaacagagctgggaaaacaagatgggagccgggaagctgacttccagttccagctgctgtttaggtctgggggagagcagatgctgggagacagtcgggggaagcgggaggtggcacgggctaggatgggccgcagcctgctctgacacggcctcttccctctccccaggtcccccgcaggcctgagtccccttcctcatctgtagacacatttgagaagccaaggtaagagaagctgaagggagagctgcaaagactgggcgtgtgagtcctgaggccaggctgtggaaccctggcttccgtcctgccttggtggggacatcctaggtaccaggagcccctcactgtgactagtgagggatgcaggggcaggcaagggtcagaggtggatgtgagcagactcttgctcctccaagaagctcagagggttctccccccaacccccactcccacccccgcccccgccttggggagcctcagccagaaccagttagttgctccttcctccatggtgggctcgcaaaactgctcttccagtaataggacaagctcagacaacggggacttgctggggacacgggtgacccctgaatgcagagctaggttttggggctcccaccgaagggaagagctcagggaggggaaggcagagtggacagatgggaggaaccagcttcttccgctcactacaggtacagaattc

SEQ ID NO: 3 - DNA, 5886 bp, Oryctolagus cuniculus
  CDS (10)...(5827)
gaattcaagatggatgactcccagatggccgactttggggcggcagcccagtacctccgcaagtccgagaaagagcgtctagaggcccagacacggcctttcgacatccgcactgagtgctttgtgcccgatgacaaggaggagttcgtcaaggccaagatcgtgtctcgggagggaggcaaggtcaccgccgaaaccgagaacggcaagacggtgaccgtgaaggaggaccaggtgttgcagcagaacccacccaagttcgacaagatcgaggacatggccatgctgaccttcctacacgagcccgctgtactctacaacctcaaggagcgctacgcggcttggatgatttacacctactcgggcctcttctgcgtcaccgtcaacccctacaagtggctgccggtgtacaatgcagaggtggtggccgcctaccggggcaagaagaggagcgaggccccgccccacatcttctccatctctgacaacgcctatcagtacatgctaacagatcgggaaaaccagtccatcctcatcaccggagaatccggggcggggaagacagtgaacaccaagcgtgtcatccagtactttgccagtattgcagccattggtgaccggggcaagaaggacaacgtcaatgccaacaagggcaccctggaggaccagatcatccaggccaaccctgccttggaggccttcggcaacgccaagaccgtccggaacgacaactcctcgcgcttcgggaaattcatcagaatccactttggagccactggaaagctggcttctgcggacatagagacctacctgctggagaagtcccgggtgatcttccagctgaaggctgagaggaactaccacatcttctaccagatcctgtccaacaagaagccggagctgctggacatgctgctgatcacaaacaacccctacgactacgccttcgtgtcccagggagaggtgtccgtggcctccatcgacgactccgaggagctcatggccaccgataacgccttcgacgtgctgggcttcagttctgaggagaaagtcggcatctacaagctgacgggcgccatcatgcactacggaaacatgaagttcaagcagaagcagcgtgaggagcaggcggagccggacggcaccgaagatgccgacaagtccgcctacctcatggggctgaactccgctgacctgctcaaggggctgtgccaccctcgggtgaaagtgggcaacgagtacgtcaccaaggggcagaacgtgcagcaggtgtactactccatcggggcgctggccaaggccgtgtacgagaagatgttcaactggatggtgatgcgcatcaatgccacgctggagaccaagctgccgcgccagtacttcataggcgtcctcgacatcgcgggcttcgagatctttgacttcaacagctttgagcagctttgcatcaacttcaccaacgagaagctgcagcagttcttcaaccaccacatgttcgtgctggagcaggaggagtacaagaaggagggcatcgagtgggagttcatcgacttcggcatggacctgcaggcctgcatcgacctcattgagaagcccatgggcatcatgtccatcctggaggaggagtgcatgttccccaaggccaccgacatgaccttcaaggccaagctatacgacaaccacctgggcaagtccaacaacttccagaagccacgcaacgtcaaggggaagcaggaagcccacttctccctggtccactacgccggcaccgtggactacaacatcctgggctggctggagaagaacaaggaccctctcaacgagacggtggtgggcctgtaccagaagtcctccctcaagctgatggccaccctcttcgccacctatgcttctgccgacactgcggacactggcaaaggcaaaggaggcaagaaaaagggctcgtccttccagacagtgtcagctctccaccgggaaaatctgaacaagctgatgaccaacctgaggaccacccaccctcacttcgtgcgctgcatcatccccaatgagcggaaggctccaggggtgatggacaaccccctggtcatgcaccagctgcgttgcaacggcgtgctggaaggcattcgcatctgcaggaagggcttccccaaccgcatcctctacggggacttccggcagaggtaccgcatcctgaacccagcggccatccctgagggccagttcattgacagcaggaagggggcggagaagctgctgggctccctggacattgaccacaaccagtacaagttcggccacaccaaggtgttcttcaaggccgggctgctggggctgctggaggagatgcgggacgagaggctgagccgcatcatcacgcgcatccaggcccagtcccggggccagctcatgcgtgctgagttcaagaagatcctggagcgcagggatgccctgctggtcatccagtggaacatccgggccttcatgggggtcaagaactggccctggatgaagctctacttcaagatcaagcctctgctgaagagcgcggagacggagaaggagatggccaccatgaaggaggagttcgggcgcatcaaagagtccctggagaagtcggaggcccgccgcaaggagctggaggagaagatggtgtcgctgctgcaggagaagaatgacctgcagctccaagtgcaggcggaacaagacaacctcaatgatgccgaggagcgctgcgaccagctgatcaagaacaagatccagctggaggccaaggtgaaggagatgaacgagaggctggaggacgaggaggagatgaacgccgagctcactgccaagaagcgcaagctggaagacgagtgctccgagctcaagaaggacattgacgacctggagctgacgctggccaaggtggagaaggagaagcacgcaaccgagaacaaggtgaagaacctgacagaggagatggctgggctggacgagatcatcgccaagctcaccaaggagaagaaagctctgcaagaggcccaccagcaggccctagatgaccttcaggctgaggaggacaaagtcaatactctgaccaaggccaagctcaagctggagcagcaggtggacgatctggagggatccctggagcaggagaagaaggtgcgcatggacctggagcgagccaagcggaagctggagggtgacctgaagctgacccaggagagcatcatggacctggagaatgacaagctgcagctggaggagaggctcaagaagaaggagtttgacatcagtcagctgaacagcaagatcgaggatgagcaggcactggccctccagctgcagaagaagctgaaggaaaaccaggcccgcatcgaggagctggaggaggagctggaggccgagcgcactgccagggccaaggtggagaagctgcgctccgacctgtcccgggagctggaggagatcagcgagcggctggaggaggccggcggggccacgtccgtgcagattgagatgaacaagaagcgcgaggccgagttccagaagatgcgacgggacctggaggaggccacgctgcaacacgaggccacggccgccgccctgcgcaagaagcacgcggacagcgtggccgagctgggcgagcagatcgacaacctgcagcgggtgaagcagaagctggaaaaggagaagagcgagttcaagctggagctggatgacgtcacctccaacatggagcagatcatcaaggccaaggcaaacctggagaaagtgtcccgcacgctggaagaccaggccaacgagtaccgcatgaagctggaggaagcccagcgctccctcaacgacttcaccacccagcgagccaagctgcaaaccgagaacggagagctagcccggcagctggaggagaaggaggcgctgatctcccagctgacccggggcaagctgtcctacacccagcagatggaggacctcaagaggcagctggaggaggaaggcaaggccaagaacgccctggcccacgcgctgcagtcggcccgccatgactgtgacctgctgcgggagcagtacgaggaggagatggaggccaaggcggagctgcagcgcgtcctgtccaaggccaactcggaggtggcacagtggaggaccaaatatgagacggacgccatccagcgcaccgaggagctggaggaggccaagaagaagctggcccagcggctgcaggacgccgaggaggccgtggaggccgtcaacgccaagtgctcctcactggagaagaccaagcaccggctgcagaacgagatcgaggacctcatggtggacgtggagcgctccaacgctgccgccgccgccctggacaagaagcagaggaacttcgacaagatcctggccgagtggaagcagaagtacgaggagtcgcagtcggagctggagtcctcgcagaaggaggcgcgctccctcagcaccgagctcttcaagctcaagaacgcctacgaggagtccctggagcacctggagaccttcaagcgggagaacaagaacctgcaggaggagatctctgacctgacggagcagctgggagaaggaggcaagaatctgcacgagctggagaaggtccgcaagcagctggaggccgagaagctggagctgcagtcggccctggaggaggccgaggcctccctggagcacgaggaaggcaagatcctccgggcccagctggagttcaaccagatcaaggcggagatcgagcggaagctggtggagaaggacgaggagatggaacaggccaagcgcaaccacctgcgggtggtggactcactgcagacctccctggatgcagagacgcgcagccgcaacgaggccctgcgggtgaagaagaagatggagggcgacctcaacgagatggagatccagctcagccaggccaacaggacggcctccgaggcccagaagcacctgaagaacgcccaagcccacctgaaggacacccagatccagctggatgacgcggtccgggccaatgacgacctgaaggagaacatcgccatcgtggagcggcgcaacgcgctgctgcaggccgagctggaggagctgcgggccgtggtggagcagacggagcggtctcggaagctggcagagcaggagctgatcgagaccagcgagcgggtgcagctgctgcactcccagaacaccagcctcatcaaccagaagaagaagatggagtcggacctgacccagctgcagacggaagtggaggaggcggtgcaggaatgcaggaacgccgaggagaaggccaagaaggccatcacggacgccgccatgatggcggaggagctgaagaaggagcaggacaccagcgcccacctggagcgcatgaagaagaacatggagcagaccattaaggacctgcagcaccggctggacgaggccgagcagatcgccctcaagggcggcaagaagcagctgcagaagctggaggcgcgggtgcgggagctggagaatgagctggaggccgagcagaagcgcaacgcggagtcggtgaagggcatgaggaagagcgagcggcgcatcaaggagctcacctaccagacggaggaggacaagaagaacctgctgcggctgcaggacctggtggacaagctgcagctgaaggtcaaggcctacaagcgccaggccgaggaggcggaggagcaggccaacaccaacctgtccaagttccgcaaggtgcagcacgagctggacgaggcggaggagcgggcagacatcgcggagtcccaggtcaacaagctgcgggccaagagccgcgacatcggcgccaagcaaaaaatgcacgacgaggagtgacgccgcctccggaaccccgctcttgttaacccgcaataaacacgagtgcctgaattc

SEQ ID NO: 4 - protein, 1939 aa, Oryctolagus cuniculus
MDDSQMADFGAAAQYLRKSEKERLEAQTRPFDIRTECFVPDDKEEFVKAKIVSREGGKVTAETENGKTVTVKEDQVLQQNPPKFDKIEDMAMLTFLHEPAVLYNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNVNANKGTLEDQIIQANPALEAFGNAKTVRNDNSSRFGKFIRIHFGATGKLASADIETYLLEKSRVIFQLKAERNYHIFYQILSNKKPELLDMLLITNNPYDYAFVSQGEVSVASIDDSEELMATDNAFDVLGFSSEEKVGIYKLTGAIMHYGNMKFKQKQREEQAEPDGTEDADKSAYLMGLNSADLLKGLCHPRVKVGNEYVTKGQNVQQVYYSIGALAKAVYEKMFNWMVMRINATLETKLPRQYFIGVLDIAGFEIFDFNSFEQLCINFTNEKLQQFFNHHMFVLEQEEYKKEGIEWEFIDFGMDLQACIDLIEKPMGIMSILEEECMFPKATDMTFKAKLYDNHLGKSNNFQKPRNVKGKQEAHFSLVHYAGTVDYNILGWLEKNKDPLNETVVGLYQKSSLKLMATLFATYASADTADTGKGKGGKKKGSSFQTVSALHRENLNKLMTNLRTTHPHFVRCIIPNERKAPGVMDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQSRGQLMRAEFKKILERRDALLVIQWNIRAFMGVKNWPWMKLYFKIKPLLKSAETEKEMATMKEEFGRIKESLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQAEEDKVNTLTKAKLKLEQQVDDLEGSLEQEKKVRMDLERAKRKLEGDLKLTQESIMDLENDKLQLEERLKKKEFDISQLNSKIEDEQALALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRMKLEEAQRSLNDFTTQRAKLQTENGELARQLEEKEALISQLTRGKLSYTQQMEDLKRQLEEEGKAKNALAHALQSARHDCDLLREQYEEEMEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNLHELEKVRKQLEAEKLELQSALEEAEASLEHEEGKILRAQLEFNQIKAEIERKLVEKDEEMEQAKRNHLRVVDSLQTSLDAETRSRNEALRVKKKMEGDLNEMEIQLSQANRTASEAQKHLKNAQAHLKDTQIQLDDAVRANDDLKENIAIVERRNALLQAELEELRAVVEQTERSRKLAEQELIETSERVQLLHSQNTSLINQKKKMESDLTQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 5 - DNA, 12801 bp, Oryctolagus cuniculus
  misc_feature 2483, 3284: n = A,T,C or G
ggatcccccctcctcggcccccagcttgctcccaggacagaacaacatggatcatgctgtgttgtcctgaaccagtctcccctggttcctggtgtgttgcctttggcacccctggatgaggggaatgcctgtgagtcaggcgccatgagatcccactgcagctgccatagagcctggctacagcaggggtcagtatggcatctggtgggctgaccaatcggaacccctctgcagggagggaggaggatgagacaggctgagctgggggctggaatggtgtcactcctggcaagtccatcagacacccatggtctgtctcagaactggctatggaaggtagcttgggctctgtgcaatctgtttgcacaaagatatatgcatacacatatgtgtctgcaaaggtaggcatgtgtgcttgcgtgcactcaaatgcatttaaggggtagggtagagctgggggtgggggacaggcagtggctctggggaggtctgcttaaggaggtagagcccttaccaaagaaatgaagtactgggggctggcactgtgtggcatagtgggtaaaagctgccacctgccatgctggcaacccatgtgggcgccagttcgagtcctggccgctccacttccgatccagctttctgccatggcctgggaaagcagtggaagatggcccaagtccttgggaccatgcacccacgtgagagacctggaagaagctcctggctcctggctttggatcggcgcagctctggccgttgtggccaatcggggagtgaatcaatgggcggaagacctctctctctctctctctctctctctctctctctctctctctccgcctctccttctctctgtataactctgactttcaaataaataaatcttaaaaaaaaaaaatgaagtcctgtaatccctttcccaggggagaagactgatgatttctcagccacttgggctcctcacaaagcaattaccttcagctctgagtaaatcccacggtctccatatcctgtgctggtccaggtgtggcggccacaggatgctgccccaagaacacctgtggacagagcacaggttctcaggaggagcagagggagcagacatctgctctgtgtgcccagcacagagacagcagtgtgcagggatcctggcttggacacaaatgcatctggcaggtgacccaaggatgctgattttccatttgcccgatgatttcacatcataacaccttggccattctccccacagagtttctagacccctgcagcctacgattgggctggctccagcgcaggtgtggatgtgactgctgaaggagcacgatcgacccagggctggatctgcccctctcctgggggcctggcttagtctgcctccgctctacagcccgatgcccctctcctgggggcctggcttagtctgcctccgctctacagcccgattcccagaccctagggccagccagctctaccagggtgcaccactagggtccctaagaccttcaggcaggacatgaaaaccagttagctggtgagtggccctaggttcccaactggtgtcctggtggatgtcaccattggacctgggccaactcctggtgggccccaagacagcactggcctgtggggagggccgttctgtgggtctcagtcaggcagtggtggccctcctggctaagggctgggtcttggagggcctgcgtaacttgggctcttgacgtgatgtttccactggcagccatgggctgggtacaggagaccataggactgcagaatacctgaccagccccggcccagaccatcccttgagccagatggacttagtgaggctgggggacaaagaactgcccccacccgccaactgcagtctgacttcagtgggttcgtgctcctgagggtgagaggagccgtttggtgagtgcctaccacgtgtcacgtgtgcactacaccctgtacccacacaatggtcacaacaaccctgggaggtaaatggaccatggccacactacagatgacaagactgtctcagagaggccctgtgactaacccagagcacatggccagaaaacggaggggctggctttcaaaacaactgaccgaagctcccaggctgcccctcacatggtcctgaggcctgcagccctggcgtccagactgcccactccccacaaagctcctccaaacactctaagatggggaagctctgtcttcccaaaccactgcaagctaaaccgagccatccctgcaactgtccactgtcgacattgtggagcagggaagcgagtgtggggtggagggccgaggtacaaaagatcttagaggccgcaggaagacaaaagcatgacaagaaggagcagctgtttgtaagggaaacttgagaccggcacaaagcccagctccctaatcccaggctcctgtggagccagggcgggcccccaaggtcagggatcctaggggcaggcagatggcagatcggcctccangcccgggtggggacccactcccatgttcacgaacaactgggggcccattgtcctttccgggttaagtccccttcctctagtgccccactctgctctcttccctttgggtttcgggggtcccttccagcttccttctttctgtgcggggtcccttttagtgtgccctacttcaccccctccccaattcctgggagtaattttagcaacttttcctctgaactgccgctggcatggtcacagcaaatggcccctgtttgggcctgacactcgatcagggtggtgacagagcccagacaaagcaggcacagagcccggggaggggggacaggagacagatcaccttccttccactccaggccttatttggtctaccttggtgaagctgccctccctccagccccttttatttatagccttttgctctgctctcctttgtcttgtcggctatcactctgcagtgccctctgcacaacccccctcctcccccctcccctccatggggccctggcccctcactctccctctaggaatgtgggtgtgtacacagggcaccagctccaagctcaggcccgaactccacttctctcaactgctgaatggagcttctcacgggaggggtctttctctggtcgctctggctagaacaatgcccagtgcgcagcaggcttccagaagcactcgctgagtgaggaagtctgcaggctgctgtccccagatggcagcaggcagaagcaagagaagcaccgtggagccaagggcaggtgctggaggggctgggacgtcagaccccactgccaccantaccaccacctaagcctggagttaggggagtgagttctattgtgcaagagggcttggagactgaacaccaatgctgatacaccgatgacccagagccttccagacttaaaaggaagcacatgaagcatgatcctttactaggtgcaaaaacagctcctgcaaagggttcaaccgaggctcaagcatcttctgtgtgtcttcattgcctgtacctgctcctgagtcctgggaactcggggaagggggcagcccccttctttgcttaggtaaccagaagggacggacgcaagggcttgctgtccatcaccagtccctgaggcccctgagtcctcggcacctgcagggcctggccaggaccctcagcgtctgcagtcagggctttcactggtgcaggctcagcttcagcagctgcaggcccacgggtcatggtccaccccacccccgcccgtctgcaccctgaacacacagtcgcttgtggttctcagacctcgccttggttaccacgcagcccccacagaagcccctcctggagtttctctccccagctgtgcccaccccaccttcaacatgtacctgatccgcaccccatcccccaggcctgcctctccttccaagctgacagccaatcagatggattaatccgcacgcgcccaagagcctcctgggaagaaggggaggggggtggatttcaaaaccaaaccacgcagcggttgcctggacacgaagccagtcaggcttgcctgcaagagccaggctgtgctctgctcagagcctgcgctccggctcccgctcctccattcccttgccctgcccgcctctcagcctcactgtagaacaccagtcgcatccaggtctgggaagaggtcacaccgctggaggaacagctgtcctggttcagggtctcctagcttagggcagctgtgcgatctcccactcctggggcgggggtcacctagtagctgggggcgcagaattgggaagacaagtcaggcgcggggctgactgtgcatcttagtcctgcgctgactaggaccatccagcactgggccccgagctctgcacacagtatgtcttccctccttcccaggactccctccacaggctgctgctatttccccgccctgattagctgggtgaccttaaacccatacttcccctctccataaaacgaaaagcttggacggataaagacctgtctggctctgggactcagtgatctcaacacatcgcctgctgatccaaggaaatcagggcctgagccagaggccccatgtcttagctccctggtcatcgtccgcgccccttagcgaagcactctccctccagctgcccccctccagcccctggttctgccctgccctccctggagctgagacttagtcccttttccatctcacagttggatgcaggctccaggccaggaaagcagggaaatttcccgttgcagaaacggggccaggtcagccttggaggctgggggcctaaggggcagcagcatgaggggcccacagtgccctgtgggagagccagggcctgcctgcctgaggtcacactggtggtcctggtcagttccctctcccacaggcagtggaatgcgaggagatattttctgctgcacgttgagccaccccgccccctggaactcagaccctgcacaccccatgccataacaatgacgaccacttccaattgtttcctggcccgagggggaggggagctctctgggagggggggccctgggggaaatgcttccagtgacaacagccctttctaaatccggctagggactgggtgcaggtgggggtgggggcgccctgctgccccatatatacaacccctgaggccaggtctggctctcagctctctcctgctctgtgtgtctttccttgatgttctcaggtaggagcggggagaagggggctccaggttaggaaggggctcccccaggaacagcaagcttcatcagggctttgtgcagagtcccaggtccggtcgttgagcacacgtgtgcaggttacacacccgtggatgtgccactgagtgtgacgctcctgtattccctggggaaggtgtctgggtctgagagtgtgggaaccaaatgaatgagtatgtgtgtgtgtgtgtgtgtgtgtgtacaagcatgtgcagtcggcaggtgagggaggggtgtgcctcactgtctgacgcagcgagtattgatgatcgtgcctgagtacgagcagaatcttttgggagtgggggggaatggtgtagaatgattgtacaggaagtgtggacgcttccaagctagtcctgagaaccatctgtccatgcacatggaggggcttgcctgtgcgtcgggagtcacgaccgctgtcgtgtgcgtgccccctgcagaaggcgctcccaggcatgtgggtgtggggcttgggtccttctacactaaagcataggcagccacatgagcctgcgtccccccacccaggcacataaacatgcggctccatagggtaccttagggggcgggttctgtggcccaggaggcggcctctgtgtgtgtgagacgtgtgcaagagcaggcatgcatggtcatgggggaggtgcagggtgctcagctgtgtggtgggtgtgtctaagagaggacacctgtcgtgtccacaaagtctgacaatgaatgggccagaaaacaactggctttggagtggggagagaggggcttgaggctcagctctaccacttaccatttatgctcttgaataagcaaatcctgtcaccttggtttccccgtctgatgcatgggatgagaactgagaggagagtttgtggactgcacagcactgagcaagtgtgaccattagctgtgtcccagatagttcaaagcaagtctcactagaaggctatgagcgtctcccaggagtgcggaaacctgttagaactggtccttggggcagcagggaacagagctgggaaaacaagatgggagccgggaagctgacttccagttccagctgctgtttaggtctgggggagagcagatgctgggagacagtcgggggaagcgggaggtggcacgggctaggatgggccgcagcctgctctgacacggcctcttccctctccccaggtcccccgcaggcctgagtccccttcctcatctgtagacacatttgagaagccaaggtaagagaagctgaagggagagctgcaaagactgggcgtgtgagtcctgaggccaggctgtggaaccctggcttccgtcctgccttggtggggacatcctaggtaccaggagcccctcactgtgactagtgagggatgcaggggcaggcaagggtcagaggtggatgtgagcagactcttgctcctccaagaagctcagagggttctccccccaacccccactcccacccccgcccccgccttggggagcctcagccagaaccagttagttgctccttcctccatggtgggctcgcaaaactgctcttccagtaataggacaagctcagacaacggggacttgctggggacacgggtgacccctgaatgcagagctaggttttggggctcccaccgaagggaagagctcagggaggggaaggcagagtggacagatgggaggaaccagcttcttccgctcactacaggtacagaattcaagatggatgactcccagatggccgactttggggcggcagcccagtacctccgcaagtccgagaaagagcgtctagaggcccagacacggcctttcgacatccgcactgagtgctttgtgcccgatgacaaggaggagttcgtcaaggccaagatcgtgtctcgggagggaggcaaggtcaccgccgaaaccgagaacggcaagacggtgaccgtgaaggaggaccaggtgttgcagcagaacccacccaagttcgacaagatcgaggacatggccatgctgaccttcctacacgagcccgctgtactctacaacctcaaggagcgctacgcggcttggatgatttacacctactcgggcctcttctgcgtcaccgtcaacccctacaagtggctgccggtgtacaatgcagaggtggtggccgcctaccggggcaagaagaggagcgaggccccgccccacatcttctccatctctgacaacgcctatcagtacatgctaacagatcgggaaaaccagtccatcctcatcaccggagaatccggggcggggaagacagtgaacaccaagcgtgtcatccagtactttgccagtattgcagccattggtgaccggggcaagaaggacaacgtcaatgccaacaagggcaccctggaggaccagatcatccaggccaaccctgccttggaggccttcggcaacgccaagaccgtccggaacgacaactcctcgcgcttcgggaaattcatcagaatccactttggagccactggaaagctggcttctgcggacatagagacctacctgctggagaagtcccgggtgatcttccagctgaaggctgagaggaactaccacatcttctaccagatcctgtccaacaagaagccggagctgctggacatgctgctgatcacaaacaacccctacgactacgccttcgtgtcccagggagaggtgtccgtggcctccatcgacgactccgaggagctcatggccaccgataacgccttcgacgtgctgggcttcagttctgaggagaaagtcggcatctacaagctgacgggcgccatcatgcactacggaaacatgaagttcaagcagaagcagcgtgaggagcaggcggagccggacggcaccgaagatgccgacaagtccgcctacctcatggggctgaactccgctgacctgctcaaggggctgtgccaccctcgggtgaaagtgggcaacgagtacgtcaccaaggggcagaacgtgcagcaggtgtactactccatcggggcgctggccaaggccgtgtacgagaagatgttcaactggatggtgatgcgcatcaatgccacgctggagaccaagctgccgcgccagtacttcataggcgtcctcgacatcgcgggcttcgagatctttgacttcaacagctttgagcagctttgcatcaacttcaccaacgagaagctgcagcagttcttcaaccaccacatgttcgtgctggagcaggaggagtacaagaaggagggcatcgagtgggagttcatcgacttcggcatggacctgcaggcctgcatcgacctcattgagaagcccatgggcatcatgtccatcctggaggaggagtgcatgttccccaaggccaccgacatgaccttcaaggccaagctatacgacaaccacctgggcaagtccaacaacttccagaagccacgcaacgtcaaggggaagcaggaagcccacttctccctggtccactacgccggcaccgtggactacaacatcctgggctggctggagaagaacaaggaccctctcaacgagacggtggtgggcctgtaccagaagtcctccctcaagctgatggccaccctcttcgccacctatgcttctgccgacactgcggacactggcaaaggcaaaggaggcaagaaaaagggctcgtccttccagacagtgtcagctctccaccgggaaaatctgaacaagctgatgaccaacctgaggaccacccaccctcacttcgtgcgctgcatcatccccaatgagcggaaggctccaggggtgatggacaaccccctggtcatgcaccagctgcgttgcaacggcgtgctggaaggcattcgcatctgcaggaagggcttccccaaccgcatcctctacggggacttccggcagaggtaccgcatcctgaacccagcggccatccctgagggccagttcattgacagcaggaagggggcggagaagctgctgggctccctggacattgaccacaaccagtacaagttcggccacaccaaggtgttcttcaaggccgggctgctggggctgctggaggagatgcgggacgagaggctgagccgcatcatcacgcgcatccaggcccagtcccggggccagctcatgcgtgctgagttcaagaagatcctggagcgcagggatgccctgctggtcatccagtggaacatccgggccttcatgggggtcaagaactggccctggatgaagctctacttcaagatcaagcctctgctgaagagcgcggagacggagaaggagatggccaccatgaaggaggagttcgggcgcatcaaagagtccctggagaagtcggaggcccgccgcaaggagctggaggagaagatggtgtcgctgctgcaggagaagaatgacctgcagctccaagtgcaggcggaacaagacaacctcaatgatgccgaggagcgctgcgaccagctgatcaagaacaagatccagctggaggccaaggtgaaggagatgaacgagaggctggaggacgaggaggagatgaacgccgagctcactgccaagaagcgcaagctggaagacgagtgctccgagctcaagaaggacattgacgacctggagctgacgctggccaaggtggagaaggagaagcacgcaaccgagaacaaggtgaagaacctgacagaggagatggctgggctggacgagatcatcgccaagctcaccaaggagaagaaagctctgcaagaggcccaccagcaggccctagatgaccttcaggctgaggaggacaaagtcaatactctgaccaaggccaagctcaagctggagcagcaggtggacgatctggagggatccctggagcaggagaagaaggtgcgcatggacctggagcgagccaagcggaagctggagggtgacctgaagctgacccaggagagcatcatggacctggagaatgacaagctgcagctggaggagaggctcaagaagaaggagtttgacatcagtcagctgaacagcaagatcgaggatgagcaggcactggccctccagctgcagaagaagctgaaggaaaaccaggcccgcatcgaggagctggaggaggagctggaggccgagcgcactgccagggccaaggtggagaagctgcgctccgacctgtcccgggagctggaggagatcagcgagcggctggaggaggccggcggggccacgtccgtgcagattgagatgaacaagaagcgcgaggccgagttccagaagatgcgacgggacctggaggaggccacgctgcaacacgaggccacggccgccgccctgcgcaagaagcacgcggacagcgtggccgagctgggcgagcagatcgacaacctgcagcgggtgaagcagaagctggaaaaggagaagagcgagttcaagctggagctggatgacgtcacctccaacatggagcagatcatcaaggccaaggcaaacctggagaaagtgtcccgcacgctggaagaccaggccaacgagtaccgcatgaagctggaggaagcccagcgctccctcaacgacttcaccacccagcgagccaagctgcaaaccgagaacggagagctagcccggcagctggaggagaaggaggcgctgatctcccagctgacccggggcaagctgtcctacacccagcagatggaggacctcaagaggcagctggaggaggaaggcaaggccaagaacgccctggcccacgcgctgcagtcggcccgccatgactgtgacctgctgcgggagcagtacgaggaggagatggaggccaaggcggagctgcagcgcgtcctgtccaaggccaactcggaggtggcacagtggaggaccaaatatgagacggacgccatccagcgcaccgaggagctggaggaggccaagaagaagctggcccagcggctgcaggacgccgaggaggccgtggaggccgtcaacgccaagtgctcctcactggagaagaccaagcaccggctgcagaacgagatcgaggacctcatggtggacgtggagcgctccaacgctgccgccgccgccctggacaagaagcagaggaacttcgacaagatcctggccgagtggaagcagaagtacgaggagtcgcagtcggagctggagtcctcgcagaaggaggcgcgctccctcagcaccgagctcttcaagctcaagaacgcctacgaggagtccctggagcacctggagaccttcaagcgggagaacaagaacctgcaggaggagatctctgacctgacggagcagctgggagaaggaggcaagaatctgcacgagctggagaaggtccgcaagcagctggaggccgagaagctggagctgcagtcggccctggaggaggccgaggcctccctggagcacgaggaaggcaagatcctccgggcccagctggagttcaaccagatcaaggcggagatcgagcggaagctggtggagaaggacgaggagatggaacaggccaagcgcaaccacctgcgggtggtggactcactgcagacctccctggatgcagagacgcgcagccgcaacgaggccctgcgggtgaagaagaagatggagggcgacctcaacgagatggagatccagctcagccaggccaacaggacggcctccgaggcccagaagcacctgaagaacgcccaagcccacctgaaggacacccagatccagctggatgacgcggtccgggccaatgacgacctgaaggagaacatcgccatcgtggagcggcgcaacgcgctgctgcaggccgagctggaggagctgcgggccgtggtggagcagacggagcggtctcggaagctggcagagcaggagctgatcgagaccagcgagcgggtgcagctgctgcactcccagaacaccagcctcatcaaccagaagaagaagatggagtcggacctgacccagctgcagacggaagtggaggaggcggtgcaggaatgcaggaacgccgaggagaaggccaagaaggccatcacggacgccgccatgatggcggaggagctgaagaaggagcaggacaccagcgcccacctggagcgcatgaagaagaacatggagcagaccattaaggacctgcagcaccggctggacgaggccgagcagatcgccctcaagggcggcaagaagcagctgcagaagctggaggcgcgggtgcgggagctggagaatgagctggaggccgagcagaagcgcaacgcggagtcggtgaagggcatgaggaagagcgagcggcgcatcaaggagctcacctaccagacggaggaggacaagaagaacctgctgcggctgcaggacctggtggacaagctgcagctgaaggtcaaggcctacaagcgccaggccgaggaggcggaggagcaggccaacaccaacctgtccaagttccgcaaggtgcagcacgagctggacgaggcggaggagcgggcagacatcgcggagtcccaggtcaacaagctgcgggccaagagccgcgacatcggcgccaagcaaaaaatgcacgacgaggagtgacgccgcctccggaaccccgctcttgttaacccgcaataaacacgagtgcctgaattc